SEQUENCE LISTING (251 sequences)

SEQ ID NO: 1 - RNA, 23 nt, Homo sapiens
agcagcauuguacagggcuauga

SEQ ID NO: 2 - RNA, 24 nt, Homo sapiens
aaaagugcuuacagugcagguagc

SEQ ID NO: 3 - RNA, 21 nt, Homo sapiens
uaaagugcugacagugcagau

SEQ ID NO: 4 - RNA, 22 nt, Homo sapiens
ucccugagacccuaacuuguga

SEQ ID NO: 5 - RNA, 18 nt, Homo sapiens
gucccuguucaggcgcca

SEQ ID NO: 6 - RNA, 17 nt, Homo sapiens
ucccuguucgggcgcca

SEQ ID NO: 7 - RNA, 24 nt, Homo sapiens
uggcccugacugaagaccagcagu

SEQ ID NO: 8 - RNA, 22 nt, Homo sapiens
cagugcaauguuaaaagggcau

SEQ ID NO: 9 - RNA, 22 nt, Homo sapiens
cagugcaaugaugaaagggcau

SEQ ID NO: 10 - RNA, 22 nt, Homo sapiens
uaacagucuacagccauggucg

SEQ ID NO: 11 - RNA, 23 nt, Homo sapiens
agcugguguugugaaucaggccg

SEQ ID NO: 12 - RNA, 22 nt, Homo sapiens
cagugguuuuacccuaugguag

SEQ ID NO: 13 - RNA, 22 nt, Homo sapiens
ugagaacugaauuccauaggcu

SEQ ID NO: 14 - RNA, 23 nt, Homo sapiens
ucuggcuccgugucuucacuccc

SEQ ID NO: 15 - RNA, 21 nt, Homo sapiens
cuagacugaagcuccuugagg

SEQ ID NO: 16 - RNA, 22 nt, Homo sapiens
uagcagcacaucaugguuuaca

SEQ ID NO: 17 - RNA, 22 nt, Homo sapiens
uagcagcacguaaauauuggcg

SEQ ID NO: 18 - RNA, 23 nt, Homo sapiens
caaagugcuuacagugcagguag

SEQ ID NO: 19 - RNA, 23 nt, Homo sapiens
caaagaauucuccuuuugggcuu

SEQ ID NO: 20 - RNA, 23 nt, Homo sapiens
uaaggugcaucuagugcagauag

SEQ ID NO: 21 - RNA, 23 nt, Homo sapiens
caacggaaucccaaaagcagcug

SEQ ID NO: 22 - RNA, 22 nt, Homo sapiens
ugggucuuugcgggcgagauga

SEQ ID NO: 23 - RNA, 22 nt, Homo sapiens
aacuggcccucaaagucccgcu

SEQ ID NO: 24 - RNA, 21 nt, Homo sapiens
uagcagcacagaaauauuggc

SEQ ID NO: 25 - RNA, 23 nt, Homo sapiens
ugugcaaaucuaugcaaaacuga

SEQ ID NO: 26 - RNA, 23 nt, Homo sapiens
ugugcaaauccaugcaaaacuga

SEQ ID NO: 27 - RNA, 22 nt, Homo sapiens
uucccuuugucauccuaugccu

SEQ ID NO: 28 - RNA, 23 nt, Homo sapiens
uaaagugcuuauagugcagguag

SEQ ID NO: 29 - RNA, 22 nt, Homo sapiens
uagcuuaucagacugauguuga

SEQ ID NO: 30 - RNA, 21 nt, Homo sapiens
uugugcuugaucuaaccaugu

SEQ ID NO: 31 - RNA, 23 nt, Homo sapiens
agcuacauugucugcuggguuuc

SEQ ID NO: 32 - RNA, 21 nt, Homo sapiens
agcuacaucuggcuacugggu

SEQ ID NO: 33 - RNA, 23 nt, Homo sapiens
caagucacuagugguuccguuua

SEQ ID NO: 34 - RNA, 22 nt, Homo sapiens
uggcucaguucagcaggaacag

SEQ ID NO: 35 - RNA, 22 nt, Homo sapiens
cauugcacuugucucggucuga

SEQ ID NO: 36 - RNA, 22 nt, Homo sapiens
uucaaguaauccaggauaggcu

SEQ ID NO: 37 - RNA, 21 nt, Homo sapiens
uucaaguaauucaggauaggu

SEQ ID NO: 38 - RNA, 21 nt, Homo sapiens
uucacaguggcuaaguuccgc

SEQ ID NO: 39 - RNA, 21 nt, Homo sapiens
uucacaguggcuaaguucugc

SEQ ID NO: 40 - RNA, 22 nt, Homo sapiens
aaggagcucacagucuauugag

SEQ ID NO: 41 - RNA, 22 nt, Homo sapiens
cacuagauugugagcuccugga

SEQ ID NO: 42 - RNA, 22 nt, Homo sapiens
uagcaccaucugaaaucgguua

SEQ ID NO: 43 - RNA, 23 nt, Homo sapiens
cagugcaauaguauugucaaagc

SEQ ID NO: 44 - RNA, 23 nt, Homo sapiens
uaagugcuuccauguuuuaguag

SEQ ID NO: 45 - RNA, 22 nt, Homo sapiens
uguaaacauccucgacuggaag

SEQ ID NO: 46 - RNA, 22 nt, Homo sapiens
uguaaacauccuacacucagcu

SEQ ID NO: 47 - RNA, 23 nt, Homo sapiens
uguaaacauccuacacucucagc

SEQ ID NO: 48 - RNA, 22 nt, Homo sapiens
uguaaacauccccgacuggaag

SEQ ID NO: 49 - RNA, 22 nt, Homo sapiens
ugcuaugccaacauauugccau

SEQ ID NO: 50 - RNA, 21 nt, Homo sapiens
aggcaagaugcuggcauagcu

SEQ ID NO: 51 - RNA, 22 nt, Homo sapiens
aaaagcuggguugagagggcga

SEQ ID NO: 52 - RNA, 23 nt, Homo sapiens
cgcauccccuagggcauuggugu

SEQ ID NO: 53 - RNA, 22 nt, Homo sapiens
cuggcccucucugcccuuccgu

SEQ ID NO: 54 - RNA, 21 nt, Homo sapiens
gccccugggccuauccuagaa

SEQ ID NO: 55 - RNA, 23 nt, Homo sapiens
ucaagagcaauaacgaaaaaugu

SEQ ID NO: 56 - RNA, 22 nt, Homo sapiens
gcugacuccuaguccagggcuc

SEQ ID NO: 57 - RNA, 22 nt, Homo sapiens
caaucagcaaguauacugcccu

SEQ ID NO: 58 - RNA, 22 nt, Homo sapiens
uggcagugucuuagcugguugu

SEQ ID NO: 59 - RNA, 23 nt, Homo sapiens
aggcaguguaguuagcugauugc

SEQ ID NO: 60 - RNA, 22 nt, Homo sapiens
uuaucagaaucuccagggguac

SEQ ID NO: 61 - RNA, 22 nt, Homo sapiens
uaaugccccuaaaaauccuuau

SEQ ID NO: 62 - RNA, 22 nt, Homo sapiens
aauugcacuuuagcaaugguga

SEQ ID NO: 63 - RNA, 22 nt, Homo sapiens
uuauaauacaaccugauaagug

SEQ ID NO: 64 - RNA, 22 nt, Homo sapiens
auauaauacaaccugcuaagug

SEQ ID NO: 65 - RNA, 23 nt, Homo sapiens
uagugcaauauugcuuauagggu

SEQ ID NO: 66 - RNA, 22 nt, Homo sapiens
aagacgggaggaaagaagggag

SEQ ID NO: 67 - RNA, 22 nt, Homo sapiens
ucaggcucaguccccucccgau

SEQ ID NO: 68 - RNA, 22 nt, Homo sapiens
aguggggaacccuuccaugagg

SEQ ID NO: 69 - RNA, 22 nt, Homo sapiens
caugccuugaguguaggaccgu

SEQ ID NO: 70 - RNA, 22 nt, Homo sapiens
ccucccacacccaaggcuugca

SEQ ID NO: 71 - RNA, 22 nt, Homo sapiens
gagcuuauucauaaaagugcag

SEQ ID NO: 72 - RNA, 22 nt, Homo sapiens
uacccauugcauaucggaguug

SEQ ID NO: 73 - RNA, 22 nt, Homo sapiens
caacaaaucacagucugccaua

SEQ ID NO: 74 - RNA, 23 nt, Homo sapiens
aaggagcuuacaaucuagcuggg

SEQ ID NO: 75 - RNA, 17 nt, Homo sapiens
ucucgcuggggccucca

SEQ ID NO: 76 - RNA, 22 nt, Homo sapiens
ugcggggcuagggcuaacagca

SEQ ID NO: 77 - RNA, 22 nt, Homo sapiens
uauugcacuugucccggccugu

SEQ ID NO: 78 - RNA, 23 nt, Homo sapiens
caaagugcuguucgugcagguag

SEQ ID NO: 79 - RNA, 22 nt, Homo sapiens
cacccguagaaccgaccuugcg

SEQ ID NO: 80 - RNA, 23 nt, Homo sapiens
uagcaccauuugaaaucaguguu

SEQ ID NO: 81 - RNA, 22 nt, Homo sapiens
cuuucagucggauguuugcagc

SEQ ID NO: 82 - RNA, 22 nt, Homo sapiens
cuuucagucggauguuuacagc

SEQ ID NO: 83 - RNA, 22 nt, Homo sapiens
aaaagcuggguugagagggcaa

SEQ ID NO: 84 - RNA, 22 nt, Homo sapiens
uaugugccuuuggacuacaucg

SEQ ID NO: 85 - RNA, 21 nt, Homo sapiens
cgcgggugcuuacugacccuu

SEQ ID NO: 86 - RNA, 23 nt, Homo sapiens
cgggucggaguuagcucaagcgg

SEQ ID NO: 87 - RNA, 22 nt, Homo sapiens
aacccguagauccgaacuugug

SEQ ID NO: 88 - RNA, 21 nt, Homo sapiens
ucagugcaugacagaacuugg

SEQ ID NO: 89 - RNA, 22 nt, Homo sapiens
cugugcgugugacagcggcuga

SEQ ID NO: 90 - RNA, 22 nt, Homo sapiens
uagcaccauuugaaaucgguua

SEQ ID NO: 91 - RNA, 21 nt, Homo sapiens
gcaguccaugggcauauacac

SEQ ID NO: 92 - RNA, 22 nt, Homo sapiens
cacgcucaugcacacacccaca

SEQ ID NO: 93 - RNA, 23 nt, Homo sapiens
uauucauuuauccccagccuaca

SEQ ID NO: 94 - RNA, 22 nt, Homo sapiens
aacccguagauccgaucuugug

SEQ ID NO: 95 - RNA, 22 nt, Homo sapiens
ugagguaguagguuguauaguu

SEQ ID NO: 96 - RNA, 22 nt, Homo sapiens
ugagguaguagguuguaugguu

SEQ ID NO: 97 - RNA, 22 nt, Homo sapiens
agagguaguagguugcauaguu

SEQ ID NO: 98 - RNA, 22 nt, Homo sapiens
ugagguaggagguuguauaguu

SEQ ID NO: 99 - RNA, 22 nt, Homo sapiens
ugagguaguagauuguauaguu

SEQ ID NO: 100 - RNA, 22 nt, Homo sapiens
ugagguaguaguuuguacaguu

SEQ ID NO: 101 - RNA, 22 nt, Homo sapiens
acagauucgauucuaggggaau

SEQ ID NO: 102 - RNA, 23 nt, Homo sapiens
uacccuguagaaccgaauuugug

SEQ ID NO: 103 - RNA, 24 nt, Homo sapiens
ucccugagacccuuuaaccuguga

SEQ ID NO: 104 - RNA, 22 nt, Homo sapiens
ucggauccgucugagcuuggcu

SEQ ID NO: 105 - RNA, 22 nt, Homo sapiens
ugugacugguugaccagagggg

SEQ ID NO: 106 - RNA, 22 nt, Homo sapiens
caucaucgucucaaaugagucu

SEQ ID NO: 107 - RNA, 23 nt, Homo sapiens
uuauugcuuaagaauacgcguag

SEQ ID NO: 108 - RNA, 21 nt, Homo sapiens
ugagaugaagcacuguagcuc

SEQ ID NO: 109 - RNA, 23 nt, Homo sapiens
guccaguuuucccaggaaucccu

SEQ ID NO: 110 - RNA, 22 nt, Homo sapiens
ugagaacugaauuccauggguu

SEQ ID NO: 111 - RNA, 22 nt, Homo sapiens
ugagaacugaauuccauaggcu

SEQ ID NO: 112 - RNA, 22 nt, Homo sapiens
aaucauacacgguugaccuauu

SEQ ID NO: 113 - RNA, 23 nt, Homo sapiens
uuaaugcuaaucgugauaggggu

SEQ ID NO: 114 - RNA, 23 nt, Homo sapiens
aacauucaacgcugucggugagu

SEQ ID NO: 115 - RNA, 22 nt, Homo sapiens
accacugaccguugacuguacc

SEQ ID NO: 116 - RNA, 22 nt, Homo sapiens
uagguaguuuccuguuguuggg

SEQ ID NO: 117 - RNA, 23 nt, Homo sapiens
cccaguguucagacuaccuguuc

SEQ ID NO: 118 - RNA, 21 nt, Homo sapiens
uaacagucuccagucacggcc

SEQ ID NO: 119 - RNA, 22 nt, Homo sapiens
acagcaggcacagacaggcagu

SEQ ID NO: 120 - RNA, 22 nt, Homo sapiens
aguucuucaguggcaagcuuua

SEQ ID NO: 121 - RNA, 22 nt, Homo sapiens
aagcugccaguugaagaacugu

SEQ ID NO: 122 - RNA, 22 nt, Homo sapiens
cucaguagccaguguagauccu

SEQ ID NO: 123 - RNA, 22 nt, Homo sapiens
acugauuucuuuugguguucag

SEQ ID NO: 124 - RNA, 21 nt, Homo sapiens
cacauuacacggucgaccucu

SEQ ID NO: 125 - RNA, 20 nt, Homo sapiens
acugccccaggugcugcugg

SEQ ID NO: 126 - RNA, 22 nt, Homo sapiens
uuuuucauuauugcuccugacc

SEQ ID NO: 127 - RNA, 21 nt, Homo sapiens
gaacggcuucauacaggaguu

SEQ ID NO: 128 - RNA, 23 nt, Homo sapiens
ugagcgccucgacgacagagccg

SEQ ID NO: 129 - RNA, 23 nt, Homo sapiens
ucccuguccuccaggagcucacg

SEQ ID NO: 130 - RNA, 22 nt, Homo sapiens
gccugcugggguggaaccuggu

SEQ ID NO: 131 - RNA, 21 nt, Homo sapiens
aucauagaggaaaauccacgu

SEQ ID NO: 132 - RNA, 21 nt, Homo sapiens
aacauagaggaaauuccacgu

SEQ ID NO: 133 - RNA, 21 nt, Homo sapiens
ugguagacuauggaacguagg

SEQ ID NO: 134 - RNA, 22 nt, Homo sapiens
gaaguuguucgugguggauucg

SEQ ID NO: 135 - RNA, 22 nt, Homo sapiens
agaucagaaggugauuguggcu

SEQ ID NO: 136 - RNA, 22 nt, Homo sapiens
gaauguugcucggugaaccccu

SEQ ID NO: 137 - RNA, 21 nt, Homo sapiens
aauauaacacagauggccugu

SEQ ID NO: 138 - RNA, 21 nt, Homo sapiens
uaguagaccguauagcguacg

SEQ ID NO: 139 - RNA, 21 nt, Homo sapiens
ugucuugcaggccgucaugca

SEQ ID NO: 140 - RNA, 23 nt, Homo sapiens
ucuuggaguaggucauugggugg

SEQ ID NO: 141 - RNA, 22 nt, Homo sapiens
aaucguacagggucauccacuu

SEQ ID NO: 142 - RNA, 22 nt, Homo sapiens
ugaaggucuacugugugccagg

SEQ ID NO: 143 - RNA, 22 nt, Homo sapiens
ugaaacauacacgggaaaccuc

SEQ ID NO: 144 - RNA, 22 nt, Homo sapiens
aaacaaacauggugcacuucuu

SEQ ID NO: 145 - RNA, 22 nt, Homo sapiens
ggagaaauuauccuuggugugu

SEQ ID NO: 146 - RNA, 22 nt, Homo sapiens
aaacauucgcggugcacuucuu

SEQ ID NO: 147 - RNA, 21 nt, Homo sapiens
acagucugcugagguuggagc

SEQ ID NO: 148 - RNA, 22 nt, Homo sapiens
uggugggccgcagaacaugugc

SEQ ID NO: 149 - RNA, 22 nt, Homo sapiens
uuugugaccugguccacuaacc

SEQ ID NO: 150 - RNA, 21 nt, Homo sapiens
uuaauaucggacaaccauugu

SEQ ID NO: 151 - RNA, 22 nt, Homo sapiens
ucguaccgugaguaauaaugcg

SEQ ID NO: 152 - RNA, 21 nt, Homo sapiens
ucacagugaaccggucucuuu

SEQ ID NO: 153 - RNA, 22 nt, Homo sapiens
uuugguccccuucaaccagcug

SEQ ID NO: 154 - RNA, 23 nt, Homo sapiens
uauggcuuuuuauuccuauguga

SEQ ID NO: 155 - RNA, 23 nt, Homo sapiens
uauggcuuuucauuccuauguga

SEQ ID NO: 156 - RNA, 22 nt, Homo sapiens
uaacacugucugguaaagaugg

SEQ ID NO: 157 - RNA, 22 nt, Homo sapiens
ucagugcacuacagaacuuugu

SEQ ID NO: 158 - RNA, 22 nt, Homo sapiens
gugaauuaccgaagggccauaa

SEQ ID NO: 159 - RNA, 22 nt, Homo sapiens
ucgugucuuguguugcagccgg

SEQ ID NO: 160 - RNA, 21 nt, Homo sapiens
cugaccuaugaauugacagcc

SEQ ID NO: 161 - RNA, 22 nt, Homo sapiens
uguaacagcaacuccaugugga

SEQ ID NO: 162 - RNA, 22 nt, Homo sapiens
uaauacugccugguaaugauga

SEQ ID NO: 163 - RNA, 23 nt, Homo sapiens
uaauacugccggguaaugaugga

SEQ ID NO: 164 - RNA, 22 nt, Homo sapiens
gugaaauguuuaggaccacuag

SEQ ID NO: 165 - RNA, 22 nt, Homo sapiens
uccuucauuccaccggagucug

SEQ ID NO: 166 - RNA, 23 nt, Homo sapiens
caaagugcucauagugcagguag

SEQ ID NO: 167 - RNA, 23 nt, Homo sapiens
cagugcaaugauauugucaaagc

SEQ ID NO: 168 - RNA, 23 nt, Homo sapiens
acuuaaacguggauguacuugcu

SEQ ID NO: 169 - RNA, 23 nt, Homo sapiens
uaagugcuuccauguuuugguga

SEQ ID NO: 170 - RNA, 22 nt, Homo sapiens
acuuuaacauggaagugcuuuc

SEQ ID NO: 171 - RNA, 22 nt, Homo sapiens
uuuaacauggggguaccugcug

SEQ ID NO: 172 - RNA, 23 nt, Homo sapiens
uaagugcuuccauguuucagugg

SEQ ID NO: 173 - RNA, 22 nt, Homo sapiens
acuuuaacauggaggcacuugc

SEQ ID NO: 174 - RNA, 23 nt, Homo sapiens
uaagugcuuccauguuugagugu

SEQ ID NO: 175 - RNA, 22 nt, Homo sapiens
uccgucucaguuacuuuauagc

SEQ ID NO: 176 - RNA, 22 nt, Homo sapiens
uuauaaagcaaugagacugauu

SEQ ID NO: 177 - RNA, 22 nt, Homo sapiens
caaucacuaacuccacugccau

SEQ ID NO: 178 - RNA, 22 nt, Homo sapiens
aauugcacgguauccaucugua

SEQ ID NO: 179 - RNA, 23 nt, Homo sapiens
aaagugcugcgacauuugagcgu

SEQ ID NO: 180 - RNA, 23 nt, Homo sapiens
ugaggggcagagagcgagacuuu

SEQ ID NO: 181 - RNA, 22 nt, Homo sapiens
gugacaucacauauacggcagc

SEQ ID NO: 182 - RNA, 22 nt, Homo sapiens
aagugcugucauagcugagguc

SEQ ID NO: 183 - RNA, 24 nt, Homo sapiens
uucuccaaaagaaagcacuuucug

SEQ ID NO: 184 - RNA, 21 nt, Homo sapiens
aaagugcuuccuuuuagaggg

SEQ ID NO: 185 - RNA, 22 nt, Homo sapiens
aaagugcuuccuuuuagagggu

SEQ ID NO: 186 - RNA, 20 nt, Homo sapiens
guccgcucggcgguggccca

SEQ ID NO: 187 - RNA, 22 nt, Homo sapiens
ugugucacucgaugaccacugu

SEQ ID NO: 188 - RNA, 22 nt, Homo sapiens
guucucccaacguaagcccagc

SEQ ID NO: 189 - RNA, 25 nt, Homo sapiens
agggaucgcgggcggguggcggccu

SEQ ID NO: 190 - RNA, 23 nt, Homo sapiens
uggaagacuagugauuuuguugu

SEQ ID NO: 191 - RNA, 22 nt, Homo sapiens
acuccagccccacagccucagc

SEQ ID NO: 192 - RNA, 22 nt, Homo sapiens
ugagaccucuggguucugagcu

SEQ ID NO: 193 - RNA, 22 nt, Homo sapiens
uccauuacacuacccugccucu

SEQ ID NO: 194 - RNA, 22 nt, Homo sapiens
auaaagcuagauaaccgaaagu

SEQ ID NO: 195 - RNA, 23 nt, Homo sapiens
ucuuugguuaucuagcuguauga

SEQ ID NO: 196 - RNA, 22 nt, Homo sapiens
acugcugagcuagcacuucccg

SEQ ID NO: 197 - RNA, 22 nt, Homo sapiens
ucuucucuguuuuggccaugug

SEQ ID NO: 198 - RNA, 22 nt, Homo sapiens
uucaacggguauuuauugagca

SEQ ID NO: 199 - RNA, 23 nt, Homo sapiens
agcagcauuguacagggcuauca

SEQ ID NO: 200 - RNA, 22 nt, Homo sapiens
uuuccggcucgcgugggugugu

SEQ ID NO: 201 - RNA, 20 nt, Homo sapiens
uaaggcacgcggugaaugcc

SEQ ID NO: 202 - RNA, 22 nt, Homo sapiens
acugcauuaugagcacuuaaag

SEQ ID NO: 203 - RNA, 22 nt, Homo sapiens
ugucaguuugucaaauacccca

SEQ ID NO: 204 - RNA, 23 nt, Homo sapiens
gaagugcuucgauuuuggggugu

SEQ ID NO: 205 - RNA, 21 nt, Homo sapiens
acuggacuuggagucagaagg

SEQ ID NO: 206 - RNA, 22 nt, Homo sapiens
cgucaacacuugcugguuuccu

SEQ ID NO: 207 - RNA, 23 nt, Homo sapiens
cacucagccuugagggcacuuuc

SEQ ID NO: 208 - RNA, 22 nt, Homo sapiens
caaagugccucccuuuagagug

SEQ ID NO: 209 - RNA, 23 nt, Homo sapiens
cucuugagggaagcacuuucugu

SEQ ID NO: 210 - RNA, 22 nt, Homo sapiens
uggucuaggauuguuggaggag

SEQ ID NO: 211 - RNA, 22 nt, Homo sapiens
gacuauagaacuuucccccuca

SEQ ID NO: 212 - RNA, 23 nt, Homo sapiens
ugugcuugcucgucccgcccgca

SEQ ID NO: 213 - RNA, 19 nt, Homo sapiens
ucuaggcugguacugcuga

SEQ ID NO: 214 - RNA, 22 nt, Homo sapiens
cugcccuggcccgagggaccga

SEQ ID NO: 215 - DNA, 23 bp, Artificial Sequence
  Primers
ttgttgttgggtttgtttttagg

SEQ ID NO: 216 - DNA, 21 bp, Artificial Sequence
  primer
accttaaacccacccctccta

SEQ ID NO: 217 - DNA, 18 bp, Artificial Sequence
  Sequencing
tttggtgagattggtaga

SEQ ID NO: 218 - DNA, 23 bp, Artificial Sequence
  Primer
aagtttttgtgggggatttgtat

SEQ ID NO: 219 - DNA, 23 bp, Artificial Sequence
  Primer
ccacccactaaccttaacctcta

SEQ ID NO: 220 - DNA, 15 bp, Artificial Sequence
  Seq 1
tgaggttttggaggg

SEQ ID NO: 221 - DNA, 25 bp, Artificial Sequence
  Seq 2
gttattattattaggtaaatatttt

SEQ ID NO: 222 - DNA, 30 bp, Artificial Sequence
  primer
agttttaggatatttaggttaggtttagaa

SEQ ID NO: 223 - DNA, 20 bp, Artificial Sequence
  primer
caacaacccccctctacaat

SEQ ID NO: 224 - DNA, 24 bp, Artificial Sequence
  Seq 1
ggatatttaggttaggtttagaaa

SEQ ID NO: 225 - DNA, 24 bp, Artificial Sequence
  Seq 2
gtatattttttaatttgttaggtt

SEQ ID NO: 226 - DNA, 18 bp, Artificial Sequence
  primer
cgcgagcacagcttcttt

SEQ ID NO: 227 - DNA, 19 bp, Artificial Sequence
  primer
tcatgaaacccgggaaagt

SEQ ID NO: 228 - DNA, 20 bp, Artificial Sequence
  primer
ctgaccggcctgtatgctat

SEQ ID NO: 229 - DNA, 21 bp, Artificial Sequence
  primer
tcagctgacagagaaacagca

SEQ ID NO: 230 - DNA, 19 bp, Artificial Sequence
  primer
gtcgtccatgtcccgagaa

SEQ ID NO: 231 - DNA, 19 bp, Artificial Sequence
  primer
gaaaccgcatcaccattcc

SEQ ID NO: 232 - DNA, 24 bp, Artificial Sequence
  primer
gagcagcactgagatcctgtttaa

SEQ ID NO: 233 - DNA, 20 bp, Artificial Sequence
  primer
aggcactggaactcgcaatg

SEQ ID NO: 234 - DNA, 24 bp, Artificial Sequence
  primer
cattcctggccctgagtcaatcgc

SEQ ID NO: 235 - DNA, 21 bp, Artificial Sequence
  primer
gtgcctgtggtcatggatctg

SEQ ID NO: 236 - DNA, 19 bp, Artificial Sequence
  primer
gagaggcagcggcagtgat

SEQ ID NO: 237 - DNA, 20 bp, Artificial Sequence
  primer
gataacgccaccgaggacaa

SEQ ID NO: 238 - DNA, 23 bp, Artificial Sequence
  primer
acctttgaagattgctcctgaga

SEQ ID NO: 239 - DNA, 20 bp, Artificial Sequence
  primer
gcagcgatatcgtcatccat

SEQ ID NO: 240 - DNA, 19 bp, Artificial Sequence
  primer
gaggtgccatcgtcaatgt

SEQ ID NO: 241 - DNA, 20 bp, Artificial Sequence
  primer
tatgttcggcttcccattct

SEQ ID NO: 242 - DNA, 20 bp, Artificial Sequence
  primer
ttgtgttgggcttccttctc

SEQ ID NO: 243 - DNA, 20 bp, Artificial Sequence
  primer
gcacaggactctcgcaatcc

SEQ ID NO: 244 - DNA, 18 bp, Artificial Sequence
  primer
tcggatggaggttggaga

SEQ ID NO: 245 - DNA, 20 bp, Artificial Sequence
  primer
atacgactcctgccctggaa

SEQ ID NO: 246 - DNA, 20 bp, Artificial Sequence
  primer
aagggcccgaacatacgatt

SEQ ID NO: 247 - DNA, 20 bp, Artificial Sequence
  primer
tggttggaaccggcatcttt

SEQ ID NO: 248 - DNA, 21 bp, Artificial Sequence
  primer
cctgtgccacttgctctttag

SEQ ID NO: 249 - DNA, 21 bp, Artificial Sequence
  primer
tgctcgatcctggatgaaacc

SEQ ID NO: 250 - DNA, 20 bp, Artificial Sequence
  primer
atggatgctctcttcgcagg

SEQ ID NO: 251 - DNA, 20 bp, Artificial Sequence
  primer
gcagcgatatcgtcatccat

(19) United States
(12) Patent Application Publication
(10) Pub. No.: US 2021/0161967 A1
(43) Pub. Date: Jun. 3, 2021

(54) EXTRACELLULAR VESICLES AND USES THEREOF

(71) Applicant: FONDAZIONE IRCCS "CA' GRANDA - OSPEDALE MAGGIORE POLICLINICO", Milano (IT)

(72) Inventors: Mario BARILANI, MILANO (IT); Lorenza LAZZARI, MILANO (IT)

(73) Assignee: FONDAZIONE IRCCS "CA' GRANDA - OSPEDALE MAGGIORE POLICLINICO", Milano (IT)

(21) Appl. No.: 16/770,230

(22) PCT Filed: Dec. 6, 2017
(86) PCT No.: PCT/IB2018/059709

§ 371 Date: Jun. 5, 2020

(30) Foreign Application Priority Data
Dec. 6, 2017 (IT) 102017000141216

(51) Int. Cl. A61K 35/28; C12N 5/0775; C12N 15/113; A61P 9/10; A61P 29/00; A61K 35/545; C12N 5/074

(52) U.S. Cl. CPC A61K 35/28; C12N 5/0665; C12N 15/113; C12N 2506/1369; A61P 29/00; A61K 35/545; C12N 5/0696; A61P 9/10

(57) ABSTRACT

The present invention refers to Extracellular Vesicles (EVs) characterized by specific panel(s) of markers, preferably miRNAs. Moreover, the present invention refers to the EVs for medical applications, preferably to treat and/or to prevent inflammation or ischemia.

### TECHNICAL FIELD

The present invention refers to Extracellular Vesicles (EVs) characterized by specific panel(s) of markers, preferably miRNAs.

Moreover, the present invention refers to the EVs for medical applications, preferably to treat and/or to prevent inflammation or ischemia.

### BACKGROUND ART

Recently, research on multipotent mesenchymal stromal cells (MSC) mechanism of action (MoA) in regenerative medicine approaches shifted its focus from secreted proteins to the generation of extracellular vesicles (EVs).

Focusing on the development of EV-based therapies, pathological conditions for which ready-to-use therapeutics and a rapid administration is essential appeared the most suitable areas of clinical application. In this context, for example, acute brain ischemia has a very limited therapeutic window of 3-6 hours for tissue plasminogen activator treatment.

This pathological condition occurs when blood is not flowing sufficiently to the brain to meet its metabolic demand, causing hypoxia, lack of nutrients, and, as a consequence, death of neural cells. Importantly, a successful therapy for acute brain ischemia could be beneficial also for other organs subject to the same risk, such as kidney, skeletal muscles, heart and lungs.

In the literature, some attempts have been disclosed to use EVs as a real, ready-to-use drug. For example, EP2736600B1 discloses microvesicles, a class of EVs, isolated from mesenchymal stem cells for use as immunosuppressive agents. WO2016/203414 discloses EVs isolated from osteoblastic lineage cells used for therapeutic treatments of bone pathologies and for diagnostic purpose.

In view of these considerations, there is a huge need to identify EVs profile or useful sources to isolate therapeutically interesting and efficient EVs, especially to treat or to prevent, acute pathologies requiring short time interventions. In particular, there is the need to identify strategies to keep the biological features, the molecular profiling and the secretome of EVs as much stable as possible in order to allow constant and consistent therapeutic approaches.

### BRIEF DESCRIPTION OF DRAWINGS

FIG. 1 shows the immunophenotype of the human induced pluripotent stem cells (hiPSCs) producing the EVs of the invention, assessed by flow cytometry, compared to that of the parental long-living cord blood-derived MSC (LL-CBMSC) and human embryonic stem cells (hESCs) as pluripotent positive control; the horizontal axis reports fluorescence intensity in a logarithmic scale, the vertical axis reports event count as percentage of maximum count for each sample. HLA-ABC, CD73 (A), CD90 and CD44 (B) are the antigens analyzed. The results show loss of MSC-specific surface markers after reprogramming to hiPSC, consistently with hESC phenotype.

FIG. 2 shows the RTqPCR analysis of hiPSCs revealing strong expression of pluripotency network-related genes, normalized to hESCs, as pluripotent positive control and compared to LL-CBMSC (A). Mean (n=3) with standard deviation is presented; ne, not expressed. Complete modification of the epigenetic landscape of NANOG and OCT4 regulatory regions after reprogramming was investigated by pyrosequencing of bisulfite-treated DNA (B); POS, position; PE, proximal enhancer; Pr, promoter. The results show acquisition of molecular traits related to pluripotency, consistently with hESC phenotype.

FIG. 3 shows flow cytometry analysis of EVs secreted by hiPSC. The analysis revealed positivity for carboxyfluorescein ester (CFSE), which produces fluorescence in the fluorescein isothiocyanate channel (FITC-A) (P2) after processing by cytoplasmic esterases, demonstrating that hiPSC-EVs are intact cytoplasm containing membrane-enclosed particles; SSC-A, side scatter.

FIG. 4 shows the characterization of the miRNome load of EVs produced by hiPSC, hESC and LL-CBMSC. The Venn diagram shows the distribution of common and unique miRNAs among the three EV stem cell sources.

FIG. 5 shows the heatmap representing the fold changes in miRNA EV incorporation comparing miRNA shared by hESC, hiPSC and LL-CBMSC; the horizontal dendrogram clusters miRNAs for similar amplification patterns, the vertical dendrogram clusters EV stem cell source for similar miRNA incorporation; the heatmap shows the fold change range (dark grey for less incorporated miRNAs, light grey for more incorporated miRNAs). The results show the differentially (UP and DOWN) and homogeneously expressed miRNA in the different sources with respect to hiPSC and hESC compared to LL-CBMSC.

FIG. 6 shows_the experimental protocol for the ex vivo assay (A). Mouse brain slices were prepared 1 week before the injury induction and they were maintained in culture in 6-well plates. Each well contained a transwell permeable to particles <400 nm in size; n=2 brain slices were used per transwell. Ischemic-like oxygen and glucose deprivation (OGD) insult consisted in a 2 hour incubation at 0.1% O₂, 0% glucose, followed by 1 hour of reoxygenation and nutrient availability. Then, LL-CBMSC co-culture or extracellular vesicle (EV) administrations were performed; EVs were administered also 24 hours post-OGD. At 48 hours post-OGD, tissue necrosis rescue was assessed (B), and samples for gene expression and secretion analysis were collected. PI, propidium iodide; CB-EV[1], one-fold dose LL-CBMSC-EV treatment; hiPSC-EV[1], one-fold dose hiPSC-EV treatment; hiPSC-EV[2], two-fold dose hiPSC-EV treatment. The results show significant reduction of necrotic cells in the treated organotypic brain slices.

FIG. 7 shows EVs of the invention PKH26-labelled and fused with target tissues. A: negative control. B: PKH26-labelled LL-CBMSC-EVs. C: PKH26-labelled hiPSC-EVs. The results show successful fusion of the EVs of the invention with the damaged tissue.

FIG. 8 shows RTqPCR analysis on neural cell type markers affected by OGD insult targeted by the different treatments (A, Map2 for neurons; B, Cd31 for endothelial cells; C, Cd11b for microglia; D, Gfap for astrocytes). The same analysis was performed for genes involved in apoptosis (E, Bcl2; F, Bax), proliferation (G, Mki67; H, Pcna), and growth factors (I, Bdnf; J, Vegf). The gene mRNA levels are presented as loge of the fold change, normalized to undamaged positive control (CTR). Statistical analysis: one-way ANOVA, followed by Tukey post-test, after normality was assessed by Kolmogorov-Smirnov test; n=6 for each experimental group; ∘∘∘p<0.001, ∘∘p<0.01, ∘p<0.05, compared to CTR; **p<0.01, *p<0.05, compared to oxygen and glucose deprivation (OGD) damaged untreated negative control; ∧p<0.05, compared to damaged brain slices in co-culture with LL-CBMSC (co-culture) condition. CB-EV[1], damaged brain slices treated with one-fold LL-CBMSC-EVs; hiPSC-EV[1], damaged brain slices treated with one-fold hiPSC-EVs; hiPSC-EV[2], damaged brain slices treated with two-fold hiPSC-EVs. The results show a complete rescue of astrocyte viability.

FIG. 9 shows protein levels of inflammatory cytokines secreted by the damaged tissue after OGD insult. Tumor necrosis factor α (TNFα) (A) or interferon γ (INFγ) (B) protein levels were normalized to undamaged positive control (CTR); mean and standard deviation is presented. Statistical analysis: one-way ANOVA, followed by Tukey post-test, after normality was assessed by Kolmogorov-Smirnov test; n=4 for CTR, n=5 for LL-CBMSC co-culture treatment (co-culture), n=6 for all other experimental conditions; ∘∘∘p<0.001, compared to CTR; ****p<0.0001; ***p<0.001; *p<0.05, compared to oxygen and glucose deprivation (OGD) damaged untreated negative control; ∧∧p<0.01, compared to the co-culture condition. CB-EV[1], one-fold dose LL-CBMSC-EV treatment; hiPSC-EV[2], two-fold dose hiPSC-EV treatment. The results show rescue of inflammatory protein levels in all treatments, significant in particular for stem cell EVs.

### DEFINITIONS

In order for the present invention to be more readily understood, certain terms are first defined below. Additional definitions for the following terms and other terms are set forth throughout the specification.

As used herewith, “Extracellular Vesicles” (EVs, or extracellular vesicles, or exosomes or vesicles) mean any membrane-enclosed/bound cytoplasm-containing bodies of cellular origin, including those of cell membrane (microvesicles/microparticles/ectosomes/shedding microvesicles) or endosomal (exosomes/nanoparticles/extracellular exosomes) origin, or resulting from the apoptotic process (apoptotic bodies/apoptotic blebs/apoptotic vesicles). In general, the interchangeably names exosomes/nanoparticles/extracellular exosomes are applied to vesicles preferably isolated by ultracentrifugation, preferably at around 100,000 xg, and/or preferably having an average size of 10-200 nm. Instead, the interchangeably names microvesicles/microparticles/ectosomes/shedding microvesicles are applied to vesicles preferably isolated by ultracentrifugation, preferably at around 10,000 xg, and/or preferably having an average size of 100-1000 nm. Finally, the interchangeably names apoptotic bodies/apoptotic blebs/apoptotic vesicles are applied to vesicles preferably isolated by ultracentrifugation, preferably at around 10,000 xg and/or having an average size of 10-5,000 nm.

As used herewith, “Cord Blood” (CB, or placental blood/umbilical cord blood) refers to the blood present in the placenta and in the umbilical cord after childbirth.

As used herewith, “Mesenchymal Stem Cells” (MSCs, or mesenchymal multipotent stem/stromal cells) refer to a stem cell type that can be isolated from several fetal, neonatal, perinatal or adult tissues, which possess multipotent differentiation properties restricted to derivatives of the mesodermal lineage and/or regenerative secretory/paracrine activity. As used herewith, “Long Living Cord Blood Mesenchymal Stem Cells” (LL-CBMSC) refer to stem cells isolated from umbilical cord blood, preferably characterized by CPD not less than 15, more preferably not less than 30.

As used herewith, “Induced Pluripotent Stem Cells” (iPSCs or human(h)iPSCs) refer to a pluripotent stem cell type artificially obtained (in-lab produced), preferably through a process called reprogramming. Pluripotent stem cells are a stem cell type (iPSC or embryonic stem cells) able to differentiate into all the cell types of the three germ layers, which constitute an adult organism. iPSC may be generated starting from stem cells having less differentiation properties and/or starting from completely differentiated cells thanks to the forced or induced expression of specific reprogramming factors, e.g. Oct4, Nanog, Lin28, Sox2, cMyc, Klf4 and any combinations of these factors. Cell reprogramming may be achieved by mean of different methodologies by using different combinations of reprogramming factors and/or by adding other supplements/molecules and/or by modifying culture conditions to improve the efficiency of the process.

In this context, all the cells used are preferably produced and available at

GMP (Good Manufacturing Practice) level and useful in clinical trials. As used herewith, “reprogramming” refers to the processes/methodologies used to generate iPSCs as defined above.

As used herewith, “cell (culture) medium” means any appropriate medium for the in vitro cultivation/growth/expansion/proliferation of cells. In particular, iPSC/pluripotent stem cell medium can be homemade with different formulations. Preferably, iPSC/pluripotent stem cell medium comprises a basal medium, preferably DMEM/F12, KO-DMEM and similar media, and/or a KO-serum replacement supplement and/or a growth factor, preferably basic FGF (bFGF), also called FGF2 and/or HBGF2 and/or BFGF and/or FGFB and/or prostatropin. Alternatively, many ready-to-use media can be used, preferably selected from: StemMACS, E7, E8, mTeSR1, mTeSR2 and Pluriton. MSC culture medium preferably comprises a basal medium, preferably aMEM, DMEM or similar media, preferably supplemented with animal serum, preferably fetal bovine serum. Said serum is preferably added at a concentration ranging from 10 to 30%. LL-CBMSC medium comprises preferably a basal medium, preferably αMEM or similar media, preferably supplemented with animal serum, preferably fetal bovine serum. In this case, serum is preferably added at a concentration ranging from 10 to 30%.

As used herewith, “ischemia” means a restriction in blood supply to cells and/or tissues and/or organs, which decreases the amount of oxygen and/or glucose requested for cellular metabolism and to maintain cells, tissues and organs viable and/or functional. If not treated, ischemia can lead to tissue death. The cause of ischemia is related to the occlusion of blood vessel by thrombus and/or fat and/or gas and/or foreign material embolisms and/or thrombosis and/or atherosclerosis and/or aneurysm and/or traumatic injury and/or vasoconstriction and/or other pathological conditions that can cause as a side effect an inadequate flow of blood in any part of the body. Ischemia refers eventually also to local anemia in any tissue that may result from congestion. In ischemia not only oxygen, but also nutrients are insufficient or lack completely. Moreover, the removal of metabolic wastes is absent. Ischemia can be partial (poor perfusion) or total.

In particular, brain ischemia (ischemic stroke) is a medical condition in which ischemia is localized in the brain. Preferably, it can be focal (affecting a specific region of the brain) or global (affecting all the brain). Specifically, acute brain ischemia refers to ischemic stroke caused by thrombolytic and/or embolic occlusion of a cerebral artery, characterized by sudden loss of blood flow to a specific region of the brain.

As used herewith, “acute” when used in connection with tissue damage and related disease, disorder, conditions, has the meaning understood by anyone skilled in the medical art. For example, it refers to a disease, disorder or condition having sudden and/or severe onset of symptoms. Typically, the term “acute” is used in contrast to the term “chronic”.

In the context of the present invention, the term “inflammation” refers to a complex biological response of cells, tissues and organs of the organism to damaging/harmful stimuli, preferably selected from: pathogens, damaged cells, and irritants. Inflammation can be acute or chronic. The former regarding the first response of the body to harmful stimuli, characterized by increased movement/migration of plasma and leukocytes from the circulation into the injured tissues. During acute inflammation, complex and coordinated biochemical events propagates and matures the inflammatory response, involving the local vascular system, the immune system, and various cells within the injured tissue. The latter regarding prolonged inflammation beyond the acute phase, which leads to a progressive shift in the type of immune cells present hi the inflamed tissue, characterized by simultaneous destruction and healing of the injured/inflamed tissue. Thus, inflammation is a protective response involving cells of the immune system, blood vessels, and many diverse molecular mediators, and its role is to remove the cause of cell injury and the necrotic cells from the damaged tissue. Depending on the diverse stimuli present at the injured tissue, inflammation can lead to either resolution of acute inflammation for the initiation of tissue repair, or to chronicization of inflammation, scarring, fibrosis impeding satisfactory of any tissue regeneration/repair.

As used herewith, “pharmaceutically acceptable carrier” refers to carriers that are approved by a regulatory agency of government or listed in the United States Pharmacopeia, the European Pharmacopeia, the United Kingdom Pharmacopeia, or other generally recognized pharmacopeia for use in animals, and in particular in humans.

As used herewith, “therapeutically effective amount” of a therapeutic agent means an amount that is sufficient, when administered to a subject suffering from or susceptible to a disease, disorder and/or condition, to treat, diagnose, prevent, and/or delay the onset of the symptoms of the disease, disorder and/or condition. It will be appreciated by those skilled in the art that a therapeutically effective amount is typically administered via dosing regimen comprising at least one unit dose.

### DETAILED DESCRIPTION OF THE INVENTION

A first aspect of the present invention refers to extracellular vesicles (EVs), preferably derived from Mesenchymal Stem Cells of Cord Blood (CB-MSCs), wherein said extracellular vesicles are characterized by the expression of a panel of miRNAs as disclosed below.

The EVs of the present invention are characterized by the expression of a panel of miRNAs comprising at least one miRNA selected from: SEQ ID NO: 1-214 and any combination thereof, preferably said panel comprises SEQ ID NO: 1-214; or said panel comprises at least one miRNA or at least one of the following group of miRNAs selected from: SEQ ID N: 1-79, SEQ ID NO: 80-86, SEQ ID NO: 87-94, SEQ ID NO: 95-150 and any combination thereof, preferably said panel comprises SEQ ID NO: 1-150; or said panel comprises at least one miRNAs or at least one of the following group of miRNAs selected from: SEQ ID NO: 1-79, SEQ ID NO: 80-86, SEQ ID NO: 151-198, SEQ ID NO: 199-214 and any combination thereof, preferably said panel comprises SEQ ID NO: 1-86, 151-214.

According to a preferred embodiment said panel of miRNAs comprises at least one, preferably all, miRNA selected from at least one of the following combination of miRNAs: the combination SEQ ID N: 1-79 and/or the combination SEQ ID NO: 80-86 and/or the combination SEQ ID NO: 87-94 and/or the combination SEQ ID NO: 95-150 and/or the combination SEQ ID NO: 151-198 and/or the combination SEQ ID NO: 199-214.

According to a further preferred embodiment of the present invention, the EVs of the present invention are characterized by the expression of a panel of miRNAs comprising at least one, preferably all, miRNA selected from: SEQ ID NO: 4, 8, 25, 164 and combination thereof which preferably target TNFα mRNA; and/or SEQ ID NO: 16, 17, 34, 37, 38, 80, 103, 108, 110, 114, 136 and combinations thereof which target INFγ mRNA.

Therefore, the miRNA(s) or the panel(s) of miRNAs represent a sort of signature(s) or fingerprint(s) of the EVs of the invention relevant also for the related medical uses here below disclosed in more detail.

Preferably, the EVs of the invention may be alternatively also named according to the definition reported above.

Generally said EVs have average size ranging from 10 to 5000 nanometers (nm), preferably 10 to 1000 nm, more preferably from 10 to 260 nm, still more preferably 10 to 150 nm. Preferably the EVs of the invention have average size ranging from 30 to 300 nanometers, more preferably from 80 to 150 nm, still more preferably their average size is around 125-135 nm.

Preferably said EVs express CD63 and/or CD81, in other words the EVs of the invention are CD63⁺ (wherein+means positive) and/or CD81⁺.

Eventually the EVs of the invention can be artificial or synthetic, preferably engineered synthetic EVs, and loaded with or expressing the panel(s) of markers/miRNAs disclosed above, and eventually further molecules and/or drugs.

As used herein, “artificial or synthetic EV” refers to chemically defined, EV-like particles loaded with a cargo of bioactive molecules. Preferably, such artificial/synthetic EVs possess equivalent and/or similar functionality/efficacy in therapeutic settings, compared to that of biologically available EVs spontaneously produced by cells in culture. Preferably, such artificial/synthetic EVs are enclosed spherical bilayer shells, with micron or submicron diameters, composed of cellular lipids or their analogs. Preferably, artificial EVs can be produced starting from defined phospholipid suspension prepared by extrusion or sonication, which is injected into an aqueous solution. In this way aqueous vesicle, solutions can be prepared of different phospholipid composition, as well as different sizes of vesicles.

The Cord Blood Mesenchymal Stem Cells (CB-MSCs) of the present invention are preferably any stem cells derived from said CB-MSCs, preferably a subtype of CB-MSCs named Long Living-Cord Blood Mesenchymal Stem Cells (LL-CB-MSCs).

Preferably, LL-CB-MSCs are immortalized cells. In this regard, any cell immortalization method can be used in this context allowing cells to start proliferating indefinitely generally by escaping the normal cellular senescence and to keep undergoing cell's division. Immortalization indeed let cells (can) be grown for prolonged periods in vitro.

The Cord Blood Mesenchymal Stem Cells (CB-MSCs) of the present invention are alternatively induced Pluripotent Stem Cells (the iPSCs as defined above) derived from said CB-MSCs by using any method able to induce pluripotency in cells, preferably by a reprogramming process.

Said LL-CB-MSCs are MSCs isolated from cord blood, preferably isolated human cord blood, preferably freshly isolated or frozen, and it is characterized by:

1) High maximum CDP (see the definition above), preferably a CDP not less than 15, more preferably a CPD not less than 30; and/or

2) Expression of at least one, preferably all, the following markers: CD90, CD105, CD73, NG2, CD146, PDGFIRβ and CD56 and/or the negative expression (they do not express) for at least one, preferably all, the following markers: CD45, CD34, and CD271. In other words preferably the LL-CB-MSCs of the invention are characterized preferably by the following immunophenotype: CD90⁺, CD105⁺, CD73⁺, NG2⁺, CD146⁺, PDGFRβ⁺, CD56⁺, CD45⁻, CD34⁻, CD271⁻ wherein (+) means positive and (−) means negative; and/or

3) The ability to produce/secrete/generate the EVs characterized by the panel of markers disclosed above.

Moreover, said LL-CB-MSCs proliferate even under low density-seeding conditions, preferably around 3-10 cells/cm², more preferably around 3 cells/cm².

According to a preferred embodiment of the invention, the LL-CB-MSCs of the invention are MSCs isolated from the cord blood by a process comprising the following steps:

(i) Obtaining an isolated sample of cord blood; and/or

(ii) Isolating mononuclear cells from the sample of cord blood, preferably by density gradient, more preferably Ficoll density gradient at a concentration preferably of about 1.077 g/mL; and/or

(iii) Removing, preferably by immunodepletion, from mononuclear cells of step (ii) at least one, preferably all, cell population selected from: lymphocytes, preferably T lymphocytes, more preferably CD3+ cells; monocytes, preferably CD14+ cells; red blood cells, preferably glycophorin A positive cells; neutrophils, preferably CD66B+ cells.

The LL-CB-MSC isolation process of the invention comprises preferably a further step (iv step) of seeding the mononuclear cells obtained by step (iii) on a tissue culture-treated plastic support. The seeding step is preferably performed in a medium comprising a standard basic medium, preferably selected from: Dulbecco's modified Eagle medium, α-modification minimal essential Eagle medium and similar media, preferably supplemented with L-glutamine and/or animal serum, preferably from bovine. Preferably, the concentration of said serum ranges between 10 and 30%. Optimized protocol to obtain more efficient (around 70-80% of processed CB units) MSC colony generation was reached seeding cells in a medium comprising a basic medium, preferably aMEM-based chemically-defined medium, comprising at least one, preferably all, the following molecules: recombinant human (rh)-bFGF, at a concentration ranging of 0.1-1 ng/ml, preferably of 0.25-0.5 ng/ml, more preferably around 0.33 ng/mL of, rh-IGF1 at a concentration ranging of 5-75 ng/mL, preferably 15-50 ng/mL, more preferably around 25 ng/mL;, clinical-grade human albumin, synthetic iron carrier, rh-insulin, nucleosides, L-glutamine, a-monothioglycerol, and synthetic lipids. Eventually, this medium is further supplemented with L-glutamine and/or animal serum, preferably from bovine in a concentration ranging from 30% to 10%. Preferably, the culture medium for the optimized protocol is SPE-IV medium (ABCell-Bio). Preferably, the seeding cell density is preferably 10²-10¹⁰, preferably 10⁴-10⁸, more preferably around 10⁶ mononuclear cells/cm².

The removing step is performed by using any method known to a man skilled in this field, preferably by immunodepletion, more preferably by using the common commercial kits based on immunomagnetic beads able to bind the specific cell population and eliminating said beads preferably by chromatography.

The support, such as the common dishes, flasks, wells used for cell culturing, is preferably a tissue culture-treated plastic.

Preferably when MSC colonies reach 60-80% confluence it is advisable to use a detachment method, preferably enzymatic, more preferably TripLE Select (Thermo Fisher Scientific), allowing a selective MSC harvest, while the remaining adherent contaminant cells, preferably selected from: endothelial-like cells, osteoclasts, osteocyte-like cells, and fibroblast-like cells, remain attached to the plastic surface.

The selectively detached MSCs are thus removed from cell suspension for further passaging and/or expansion.

Therefore, a further aspect of the present invention refers to isolated LL-CBMSCs obtainable/obtained by applying the process disclosed above and preferably characterized by:

1) High CDP (see the definition above), preferably CDP value not less than 15, preferably not less than 25, more preferably around 30; and/or

2) The expression of at least one, preferably all, the following markers: CD90, CD105, CD73, NG2, CD146, PDGFIRβ and CD56 and/or the negative expression (they do not express) for at least one, preferably all, the following markers: CD45, CD34, and CD271. In other words preferably the isolated LL-CBMSCs obtainable/obtained by using the method of the invention are characterized preferably by the following immunophenotype: CD90⁺, CD105⁺, CD73⁺, NG2⁺, CD146⁺, PDGFRβ⁺ CD56⁺, CD45⁻, CD34⁻, CD271⁻ wherein (+) means positive and (−) means negative; and/or

3) The ability to produce/secrete/generate the EVs characterized by the panel of markers disclosed above.

Moreover, said LL-CB-MSCs because of their CDP and lifespan (preferably more than 8 passages in culture), may be also defined clonogeneic meaning that they are able to proliferate under low density-seeding conditions, preferably the proliferate even when seed at around 3-10 cells/cm², more preferably around 3 cells/cm², generating new LL-CB-MSC colonies.

EVs are isolated starting from LL-CB-MSCs after a starvation step that is culturing cells in a serum-free culture medium/solution for a determined time in order to remove the EVs derived from the serum generally contained in the culture medium of the LL-CB-MSCs. The starvation of the cells is performed preferably when LL-CB-MSCs reached 75-90% confluence.

The EVs are isolated from the serum-free conditioned medium collected after the starvation step. Preferably, LL-CB-MSCs are cultured after the starvation step in a serum-free medium for at least 18-24 hours.

The EVs are collected preferably by ultracentrifuging the serum-free conditioned medium, preferably after dead cells and/or debris removal. Further methods of EV's collection that can be used in alternative or combination are selected from: tangential flow filtration, precipitation with volume-excluding polymers, size-exclusion chromatography, and density gradient centrifugation.

The dead cells and/or debris removal is preferably performed by centrifuging the serum-free conditioned medium collected after the starvation step for at least few minutes until 15-30 minutes at 350-5,000 g. The ultracentrifuging step of the serum-free conditioned medium collected after the starvation step is preferably performed for 1-24 hours, preferably for about 3 hours at 80,000-150,000 g at low temperature, preferably at about 4° C.

The induced Pluripotent Stem Cells (iPSCs) of the invention are obtained starting from MSCs of cord blood, preferably from the LL-CB-MSCs obtained as disclosed above in detail. In other words, Pluripotent Stem Cells are induced preferably by reprogramming MSCs from cord blood, more preferably from the LL-CB-MSCs obtained as disclosed above in detail.

According to a preferred embodiment, iPSCs are characterized by ability to produce/secrete/generate the EVs characterized by the panel of markers disclosed above.

In this regard, any methods useful to induce pluripotency in cells can be used.

The reprogramming allows transforming any adult, differentiated (somatic/mature) cells into pluripotent stem cells, that are cells able to differentiate into almost any cell in the body or in any case, the cells derived from the three germ layers. In this context the reprogramming refers to an extraembryonic perinatal tissue comprising an intermediate cell type which has recently been described to combine qualities of both the adult cells and the ESCs and possess immunoprivileged characteristics, as well as a broad multipotent plasticity.

Any method known in the art to reprogram adult/perinatal cells can be used in this context.

According to a preferred embodiment of the invention, said MSCs from cord blood, preferably the LL-CB-MSCs obtained as disclosed above in detail, are reprogrammed by the introduction at least one of, preferably all, the following reprogramming factors OCT4, SOX2, KLF4, cMYC and any combination thereof.

Preferably, the introduction of the gene codifying the reprogramming factor(s) is performed by using a non-integrating viral-based system.

This system is zero-fingerprint because no exogenous DNA is integrated into the host genome, the vector remains in the cytoplasm and consequently the genetic information of the reprogrammed cell is not altered. Moreover, one infection is generally requested in order to obtain a successful reprogramming compared to viral-free reprogramming methods.

Therefore, a further aspect of the present invention refers to induced Pluripotent Stem Cells (iPSCs), obtained/obtainable by using the method disclosed above. Preferably said iPSCs is the cell line CBMSC304-hiPSC—deposited on Oct. 26th 2017 at the Leibniz-lnstitut DSMZ-Deutsche Sammlung von Mikroorganlsmen and Zellkulturen GmbH with Accession Number DSM ACC3332 according to the provisions of the Budapepest Treaty.

According to a preferred embodiment, the iPSCs, preferably the cells of the line DSMACC3332, are characterized by ability to produce/secrete/generate the EVs characterized by the panel of markers disclosed above.

The EVs isolation process from iPSCs culturing medium is performed as already disclosed above for LL-CB-MSCs with the exception of the starvation step, since the culturing medium of these cells do not contain serum (it is a serum-free medium) and therefore, this step may not be requested.

Generally, in the standard culturing condition disclosed above, preferably with reference to the iPSCs, the average number of EVs released by the cells in the conditioned medium ranges from 1 to 50 billion, per 100 mm Petri dish per day.

As used herein, “conditioned” means modified with respect to its original composition, therefore, “conditioned medium” refers to a cell culture medium which is modified in its original composition by the metabolic activities of cells cultured in that cell culture medium. Thus, conditioned medium is already partially used by cells in culture and therefore is depleted of some of its components. Yet, it is enriched with cell-derived material, including growth factors and EVs. Conditioned media are generally used to support the growth of specific cell types. More recently, conditioned media are used to isolate EVs released by cells in the cell culture medium.

Therefore, a further aspect of the present invention refers to EVs obtainable/obtained from the LL-CB-MSCs and/or from the iPSCs reported above and characterized by a panel of miRNAs comprising at least one miRNA, preferably all miRNAs, selected from: SEQ ID NO: 1-214 and any combination thereof, preferably said panel of miRNAs comprises at least one , preferably all, miRNA selected from at least one: SEQ ID N: 1-79, and/or SEQ ID NO: 80-86, SEQ ID NO: 87-94, SEQ ID NO: 95-150 and any combination thereof, or SEQ ID NO: 1-79, SEQ ID NO: 80-86, SEQ ID NO: 151-198, SEQ ID NO: 199-214 and any combination thereof, more preferably said panel of miRNAs comprises at least one, preferably all, miRNA selected from at least one of the following combination of miRNAs: the combination SEQ ID N: 1-79 and/or the combination SEQ ID NO: 80-86 and/or the combination SEQ ID NO: 87-94 and/or the combination SEQ ID NO: 95-150 and/or the combination SEQ ID NO: 151-198 and/or the combination SEQ ID NO: 199-214 wherein: SEQ ID N: 1-79 are expressed in EVs (obtained/obtainable) from both the LL-CB-MSCs and iPSCs reported above and from standard human ESCs; SEQ ID NO: 80-86 are expressed in EVs (obtained/obtainable) from both the LL-CB-MSCs and iPSCs reported above; SEQ ID NO: 87-94 are expressed in EVs (obtained/obtainable) from LL-CB-MSCs and from standard human ESCs; SEQ ID NO: 95-150 are expressed in EVs (obtained/obtainable) from LL-CB-MSCs reported above; SEQ ID NO: 151-198 are expressed in EVs (obtained/obtainable) from iPSCs reported above and from standard human ESCs; SEQ ID NO: 199-214 are expressed in EVs (obtained/obtainable) from iPSCs reported above.

According to a preferred embodiment, the EVs are characterized by the expression of a panel of miRNAs comprising at least one miRNA, preferably all miRNAs, selected from: SEQ ID NO: 2, 4, 6, 8, 16, 17, 18, 25, 26, 28, 29, 31, 32, 34, 37, 38, 44, 50, 62, 77, 80, 87, 94, 103, 108, 110, 114, 136, 164, 169, 172, 174 and combinations thereof. Preferably the EVs are characterized by the expression of a panel of miRNAs comprising at least one miRNA, preferably at least 5 or all miRNAs, selected from: SEQ ID NO: 4, 6, 28, 29, 31, 32, 34, 50, 87, 94 and combinations thereof wherein said EVs are from LL-CB-MSCs as disclosed herewith. Preferably the EVs are characterized by the expression of a panel of miRNAs comprising at least one miRNA, preferably at least 5 or all miRNAs, selected from: SEQ ID NO: 2, 18, 26, 28, 44, 62, 77, 169, 172, 174 and combinations thereof wherein said EVs are from the iPSCs as disclosed herewith. Preferably the EVs are characterized by the expression of a panel of miRNAs comprising at least one miRNA, preferably at least 3 or all miRNAs, selected from: SEQ ID NO: 4, 8, 16, 17, 25, 34, 37, 38, 80, 103, 108, 110, 114, 136, 164 and combinations thereof wherein said miRNAs targeting TNFα and/or INFγ mRNA(s).

According to a preferred embodiment of the present invention, the EVs are characterized by the expression of a panel of miRNAs comprising at least one miRNA, preferably all miRNAs, selected from: SEQ ID NO: 4, 8, 25, 164 and combination thereof which preferably target TNFα mRNA; and/or SEQ ID NO: 16, 17, 34, 37, 38, 80, 103, 108, 110, 114, 136 and combinations thereof which target INFγ mRNA.

Generally said EVs have average size ranging from 10 to 5000 nanometers (nm), preferably 10 to 1000 nm, more preferably from 10 to 260 nm, still more preferably 10 to 150 nm. Preferably the EVs of the invention have average size ranging from 30 to 300 nanometers, more preferably from 80 to 150 nm, still more preferably their average size is around 125-135 nm.

Preferably said EVs express CD63 and/or CD81, in other words the EVs of the invention are CD63⁺ (that is CD63 positive) and/or CD81⁺.

Eventually the EVs of the invention can be artificial or synthetic, preferably engineered synthetic EVs, and loaded with the panel of markers disclosed above eventually further molecules and/or drugs.

A further aspect of the present invention refers to a signature or a panel of markers said markers being at least one miRNA, preferably all miRNAs, selected from: SEQ ID NO: 1-214 and any combination thereof, preferably said panel of miRNAs comprises at least one , preferably all, miRNA selected from at least one: SEQ ID N: 1-79, and/or SEQ ID NO: 80-86, SEQ ID NO: 87-94, SEQ ID NO: 95-150 and any combination thereof, or SEQ ID NO: 1-79, SEQ ID NO: 80-86, SEQ ID NO: 151-198, SEQ ID NO: 199-214 and any combination thereof, more preferably said panel of miRNAs comprises at least one, preferably all, miRNA selected from at least one of the following combination of miRNAs: the combination SEQ ID N: 1-79 and/or the combination SEQ ID NO: 80-86 and/or the combination SEQ ID NO: 87-94 and/or the combination SEQ ID NO: 95-150 and/or the combination SEQ ID NO: 151-198 and/or the combination SEQ ID NO: 199-214 wherein: SEQ ID N: 1-79 are expressed in EVs (obtained/obtainable) from both the LL-CB-MSCs and iPSCs reported above and from standard human ESCs; SEQ ID NO: 80-86 are expressed in EVs (obtained/obtainable) from both the LL-CB-MSCs and iPSCs reported above; SEQ ID NO: 87-94 are expressed in EVs (obtained/obtainable) from LL-CB-MSCs and from standard human ESCs; SEQ ID NO: 95-150 are expressed in EVs (obtained/obtainable) from LL-CB-MSCs reported above; SEQ ID NO: 151-198 are expressed in EVs (obtained/obtainable) from iPSCs reported above and from standard human ESCs; SEQ ID NO: 199-214 are expressed in EVs (obtained/obtainable) from iPSCs reported above.

In a preferred embodiment of the present invention, said panel of markers comprises at least one miRNA, preferably all miRNAs, selected from: comprising at least one, preferably all, miRNA selected from: SEQ ID NO: 4, 8, 25, 164 and combination thereof which preferably target TNFα mRNA; and/or SEQ ID NO: 16, 17, 34, 37, 38, 80, 103, 108, 110, 114, 136 and combinations thereof which target INFγ mRNA.

Preferably, said signature(s) is spotted or bounded/linked, preferably chemically bounded/linked, on a chip for microarray analysis. In alternative, signature(s) specific primers are spotted or deposited or bounded/linked, preferably chemically bounded/linked, on a chip for PCR-array analysis. In alternative, signature(s) specific primers are prepared as a component of a reaction mix for PCR analysis. In alternative, signature(s) specific oligonucleotides are loaded or bounded/linked, preferably chemically bounded/linked, on beads or chromatographic columns for selection and/or detection studies.

Eventually, said sequences/miRNAs can be used also as markers/probes. A further aspect of the present invention refers to a pharmaceutical composition comprising the EVs of the present invention—from LL-CB-MSCs and/or from iPSCs—and further pharmaceutically acceptable excipients/adjuvants/carriers as defined above.

Preferably said composition comprises all excipients/adjuvants/carriers standardly used for topical, enteral or parenteral route of administration, such as protective agents against enzymatic digestion in physiological or pathological contexts, bio-compatible substances to encapsulate or lyophilize EVs, synthetic lipid-based systems to deliver EVs in multi-vesicular lyposome bodies, spray formulations for oral/respiratory system use, polymer-based degradable/not degradable gels.

According to a preferred embodiment, the pharmaceutical composition as well as the EVs of the invention is/are frozen.

A further aspect of the present invention refers to the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above for use as a medicament, preferably for use in the treatment and/or prevention and/or follow-up of ischemia, preferably tissue and/or organ ischemia.

The EVs as disclosed above for the medical applications here disclosed (the EV-based therapeutic treatments) work as a cell-free system that helps the ischemic area, tissue and/or organ, to regenerate and/or repair. A further aspect of the present invention refers to the use of the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above for use in regenerative medicine, preferably to regenerate and/or to stimulate and/or to repair a body area, preferably a tissue and/or an organ, preferably after ischemia.

The therapeutic/curative/preventive efficacy of the cells/EVs/signature(s)/composition comprising EVs of the invention is correlated to the specific and unique molecular profile or cargo disclosed above. Moreover, the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above are particularly effective and useful because they elicit a prompt response. Therefore, they are particularly indicated when a prompt action is requested such as after an ischemia indeed, preferably a stroke, where an action is generally beneficial in the first 3-4 hours after the insult/injury.

A further advantage of the EVs/composition comprising EVs of the invention compared to the use of a cell-based system is that they are ready-to-use and less vulnerable to freezing procedure and/o to shear stress. In addition, they are a safer therapeutic agent than cell therapy products.

These advantages are particularly key for medical purposes, as everybody well know.

Moreover, the EVs and/or the pharmaceutical composition of the invention are particularly advantageous because they show a privileged access to the Blood Brain Barrier (BBB) and this feature is particularly key for medical purposes, especially for therapeutic approaches targeting the central nervous system.

Preferably, the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above are also useful to treat the side effects and/or symptoms and/or any drawback caused by and/or associated with ischemia or ischemic events.

Said side effects and/or symptoms and/or any drawback are preferably selected from: secondary brain injury and/or reperfusion injury.

According to a preferred embodiment of the invention, the ischemia affect any tissue, preferably a tissue selected from: connective tissue, vasculature, epithelium, endothelium, the nervous tissue, preferably all the cells forming the aforementioned tissues with respect to both parenchymal and stromal cellular components, more preferably neurons and/or glial/neuroglial cells, preferably astrocytes, oligodendrocytes, microglia, ependymal cells. Astrocytes are the most preferred cells that benefit from therapeutic treatments based on the use of the EVs/composition of the invention, preferably after brain ischemia, preferably acute brain ischemia as well demonstrated in the example below.

According to a preferred embodiment of the invention, the ischemia affect any organ, preferably selected from: brain, pancreas, intestine, skeletal muscles, heart, lung, limbs, kidney, liver, eyes, skin, spleen, thymus, bones, tendons, musculoskeletal grafts, corneae, heart valves, nerves, veins, the central and/or peripheral nervous system, more preferably the brain. Preferably said organ is useful for transplantations and it shows ischemic and/or reperfusion damages, preferably because of the organ's explantation and/or the organ's transport and/or the organ's transplantation itself or implantation.

Preferably, said ischemia is acute and/or chronic.

According to a further aspect of the present invention the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above are/is used in combination or in association with further molecules wherein said molecules are selected from: anticancer molecules, antinflammatory molecules, antibiotics, anticoagulants, antiplatelet agents, antihypertensive drugs, insulin, antipyretics, thrombolytics, and all present and new molecules adopted in the treatment of ischemic stroke.

A further aspect of the present invention, refers to a method to treat and/or to prevent an ischemia, preferably a tissue and/or an organ ischemia, in an individual said method comprising at least one step of administering to said individual an effective amount of the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above.

Preferably said administering step is performed before and/or after the ischemia affected said individual.

Preferably, the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above are also useful to treat the side effects and/or symptoms and/or any drawback caused by and/or associated with ischemia or ischemic events.

Said side effects and/or symptoms and/or any drawback are preferably selected from: secondary brain injury, reperfusion injury.

According to a preferred embodiment of the invention, the ischemia affect any tissue, preferably a tissue selected from: connective tissue, vasculature, epithelium, endothelium, the nervous tissue, preferably all the cells forming the aforementioned tissues with respect to both parenchymal and stromal cellular components, more preferably neurons and/or glial/neuroglial cells, preferably astrocytes, oligodendrocytes, microglia, ependymal cells. Astrocytes are the most preferred cells that benefit from therapeutic treatments based on the use of the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above, preferably after brain ischemia, preferably acute brain ischemia as well demonstrated in the example below.

According to a preferred embodiment of the invention, the ischemia affect any organ, preferably selected from: brain, pancreas, intestine, skeletal muscles, heart, lung, limbs, kidney, liver, eyes, skin, spleen, thymus, bones, tendons, musculoskeletal grafts, corneae, heart valves, nerves, veins, the central and/or peripheral nervous system, more preferably the brain. According to a preferred embodiment of the invention, said injury/damage affect(s) any tissue and/or organ used for transplantation in patients, preferably a tissue and/or an organ selected from: lung, heart, liver, spleen, kidney, intestine, limbs, skin, bones, thymus, tendons, musculoskeletal grafts, corneae, heart valves, nerves and veins. Preferably said organ is useful for transplantations and it shows ischemic and/or reperfusion damages, preferably because of the organ's explant and/or the organ's transport and/or the organ's transplantation itself or implantation.

Preferably, said ischemia is acute and/or chronic.

According to a preferred embodiment of the invention, said ischemia is an acute brain ischemia.

According to a further aspect of the present invention the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above are/is used in combination or in association with further molecules wherein said molecules are selected from: anticancer molecules, antinflammatory molecules, antibiotics, anticoagulants, antiplatelet agents, antihypertensive drugs, insulin, antipyretics, thrombolytics, and all present and new molecules adopted in the treatment of ischemic stroke.

A further aspect of the present invention refers to the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above for use to treat and/or to prevent an inflammation, preferably an inflammation response.

A further aspect of the present invention refers to a method to treat and/or to prevent an inflammation, preferably an inflammation response in an individual said method comprising at least one step of administering to said individual an effective amount of the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above.

Preferably said inflammation affects a tissue and/or an organ as defined before. Preferably said inflammation is in response to a damaging stimulus, preferably said damaging stimulus being selected from: infections by pathogens, necrosis, trauma, physical injury, chemical irritants, burns, frostbite, ionizing radiations, immune reactions due to hypersensitivity, stress, toxins, alcohol, psychological excitement.

Preferably said organ is selected from: brain, pancreas, intestine, skeletal muscles, heart, lung, limbs, kidney, liver, eyes, skin, spleen, thymus, bones, tendons, musculoskeletal grafts, corneae, heart valves, nerves, veins, the central and/or peripheral nervous system, more preferably the brain; and/or said tissue is preferably selected from: connective tissue, vasculature, epithelium, endothelium, the nervous tissue, preferably all the cells forming the aforementioned tissues with respect to both parenchymal and stromal cellular components, more preferably neurons and/or glial/neuroglial cells, preferably astrocytes, oligodendrocytes, microglia, ependymal cells.

Preferably said inflammation response is mediated by TNFα and/or IGFγ.

Preferably said administering step is performed before and/or after the inflammation affected said individual.

In a preferred embodiment of the present invention, for this specific purpose, treatment/prevention of inflammation, preferably acute inflammation particularly useful are EVs characterized by the expression of a panel of miRNAs comprising at least one, preferably all, mlRNA selected from: SEQ ID NO: 4, 8, 25, 164 and combination thereof which preferably target TNFα mRNA; and/or SEQ ID NO: 16, 17, 34, 37, 38, 80, 103, 108, 110, 114, 136 and combinations thereof which target INFγ mRNA.

Preferably, the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above are also useful to treat the side effects and/or symptoms and/or any drawback caused by and/or associated with said inflammation. Said side effects and/or symptoms and/or any drawback are preferably selected from: tissue pain, tissue heat, tissue redness, tissue swelling, tissue loss of function, release of inflammatory mediators, vasodilation, increased vessel permeability, recruitment of immune system cells, migration of immune system cells, activation of the complement system, systemic inflammation and any combination thereof.

According to a preferred embodiment of the invention, the inflammation affects any tissue and/or organ of the body, preferably any vascularized tissue/organ.

Preferably, said inflammation is acute and/or chronic.

According to a further aspect of the present invention the LL-CBMSCs and/or the iPSCs as disclosed above, preferably the cell line DSM ACC3332, and/or the EVs from said cells as disclosed above, and/or the signature(s) as disclosed above, and/or the pharmaceutical composition comprising said EVs as disclosed above are/is used in combination or in association with further molecules wherein said molecules are selected from: anticancer molecules, antinflammatory molecules, antibiotics, anticoagulants, antiplatelet agents, antihypertensive drugs, insulin, antipyretics, thrombolytics, and all present and new molecules adopted in the treatment of ischemic stroke.

A further aspect of the present invention refers to a kit comprising the LL-CBMSCs and/or the iPSCs and/or EVs from said cells and/or a pharmaceutical composition comprising said EVs and/or the signature(s) disclosed above.

Preferably, said kit is useful for medical purposes, preferably for the medical indications disclosed in more detail above.

The sequences disclosed in the present invention are listed in Table I and are provided herewith as SequenceListing. Any sequences having 80-99.9% of sequence identity with the listed sequences should be considered also as part of the present disclosure.

TABLE I

| SEQ ID NO | SEQUENCE | NAME/miRBase ID |
|---|---|---|
| SEQ ID NO: 1 | AGCAGCAUUGUACAGGGCUAUGA | hsa-miR-103/hsa- |
| | | miR-103a-3p |
| SEQ ID NO: 2 | AAAAGUGCUUACAGUGCAGGUAGC | hsa-miR-106a/hsa- |
| | | miR-106a-5p |
| SEQ ID NO: 3 | UAAAGUGCUGACAGUGCAGAU | hsa-miR-106b/hsa- |
| | | miR-106b-5p |
| SEQ ID NO: 4 | UCCCUGAGACCCUAACUUGUGA | hsa-miR-125b/hsa- |
| | | miR-125b-5p |
| SEQ ID NO: 5 | GUCCCUGUUCAGGCGCCA | hsa-miR-1274A/hsa- |
| | | miR-1274a |
| SEQ ID NO: 6 | UCCCUGUUCGGGCGCCA | hsa-miR-1274B/hsa- |
| | | miR-1274b |
| SEQ ID NO: 7 | UGGCCCUGACUGAAGACCAGCAGU | hsa-miR-1291 |
| SEQ ID NO: 8 | CAGUGCAAUGUUAAAAGGGCAU | hsa-miR-130a/hsa- |
| | | miR-130a-3p |
| SEQ ID NO: 9 | CAGUGCAAUGAUGAAAGGGCAU | hsa-miR-130b/hsa- |
| | | miR-130b-3p |
| SEQ ID NO: 10 | UAACAGUCUACAGCCAUGGUCG | hsa-miR-132/hsa- |
| | | miR-132-3p |
| SEQ ID NO: 11 | AGCUGGUGUUGUGAAUCAGGCCG | hsa-miR-138/hsa- |
| | | miR-138-5p |
| SEQ ID NO: 12 | CAGUGGUUUUACCCUAUGGUAG | mmu-miR-140/hsa- |
| | | miR-140-5p |
| SEQ ID NO: 13 | UGAGAACUGAAUUCCAUAGGCU | hsa-miR-146b/hsa- |
| | | miR-146b-5p |
| SEQ ID NO: 14 | UCUGGCUCCGUGUCUUCACUCCC | hsa-miR-149/hsa- |
| | | miR-149-5p |
| SEQ ID NO: 15 | CUAGACUGAAGCUCCUUGAGG | hsa-miR-151-3p/hsa- |
| | | miR-151a-3p |
| SEQ ID NO: 16 | UAGCAGCACAUCAUGGUUUACA | hsa-miR-15b/hsa- |
| | | miR-15b-5p |
| SEQ ID NO: 17 | UAGCAGCACGUAAAUAUUGGCG | hsa-miR-16/hsa-miR- |
| | | 16-5p |
| SEQ ID NO: 18 | CAAAGUGCUUACAGUGCAGGUAG | hsa-miR-17/hsa-miR- |
| | | 17-5p |
| SEQ ID NO: 19 | CAAAGAAUUCUCCUUUUGGGCUU | hsa-miR-186/hsa- |
| | | miR-186-5p |
| SEQ ID NO: 20 | UAAGGUGCAUCUAGUGCAGAUAG | hsa-miR-18a/hsa- |
| | | miR-18a-5p |
| SEQ ID NO: 21 | CAACGGAAUCCCAAAAGCAGCUG | hsa-miR-191/hsa- |
| | | miR-191-5p |
| SEQ ID NO: 22 | UGGGUCUUUGCGGGCGAGAUGA | hsa-miR-193a-5p |
| SEQ ID NO: 23 | AACUGGCCCUCAAAGUCCCGCU | hsa-miR-193b/hsa- |
| | | miR-193b-3p |
| SEQ ID NO: 24 | UAGCAGCACAGAAAUAUUGGC | hsa-miR-195/hsa- |
| | | miR-195-5p |
| SEQ ID NO: 25 | UGUGCAAAUCUAUGCAAAACUGA | hsa-miR-19a/hsa- |
| | | miR-19a-3p |
| SEQ ID NO: 26 | UGUGCAAAUCCAUGCAAAACUGA | hsa-miR-19b/hsa- |
| | | miR-19b-3p |
| SEQ ID NO: 27 | UUCCCUUUGUCAUCCUAUGCCU | hsa-miR-204/hsa- |
| | | miR-204-5p |
| SEQ ID NO: 28 | UAAAGUGCUUAUAGUGCAGGUAG | hsa-miR-20a/hsa- |
| | | miR-20a-5p |
| SEQ ID NO: 29 | UAGCUUAUCAGACUGAUGUUGA | hsa-miR-21/hsa-miR- |
| | | 21-5p |
| SEQ ID NO: 30 | UUGUGCUUGAUCUAACCAUGU | hsa-miR-218/hsa- |
| | | miR-218-5p |
| SEQ ID NO: 31 | AGCUACAUUGUCUGCUGGGUUUC | hsa-miR-221/hsa- |
| | | miR-221-3p |
| SEQ ID NO: 32 | AGCUACAUCUGGCUACUGGGU | hsa-miR-222/hsa- |
| | | miR-222-3p |
| SEQ ID NO: 33 | CAAGUCACUAGUGGUUCCGUUUA | hsa-miR-224/hsa- |
| | | miR-224-5p |
| SEQ ID NO: 34 | UGGCUCAGUUCAGCAGGAACAG | hsa-miR-24/hsa-miR- |
| | | 24-3p |
| SEQ ID NO: 35 | CAUUGCACUUGUCUCGGUCUGA | hsa-miR-25/hsa-miR- |
| | | 25-3p |
| SEQ ID NO: 36 | UUCAAGUAAUCCAGGAUAGGCU | hsa-miR-26a/hsa- |
| | | miR-26a-5p |
| SEQ ID NO: 37 | UUCAAGUAAUUCAGGAUAGGU | hsa-miR-26b/hsa- |
| | | miR-26b-5p |
| SEQ ID NO: 38 | UUCACAGUGGCUAAGUUCCGC | hsa-miR-27a/hsa- |
| | | miR-27a-3p |
| SEQ ID NO: 39 | UUCACAGUGGCUAAGUUCUGC | hsa-miR-27b/hsa- |
| | | miR-27b-3p |
| SEQ ID NO: 40 | AAGGAGCUCACAGUCUAUUGAG | hsa-miR-28/hsa-miR- |
| | | 28-5p |
| SEQ ID NO: 41 | CACUAGAUUGUGAGCUCCUGGA | hsa-miR-28-3p |
| SEQ ID NO: 42 | UAGCACCAUCUGAAAUCGGUUA | hsa-miR-29a/hsa- |
| | | miR-29a-3p |
| SEQ ID NO: 43 | CAGUGCAAUAGUAUUGUCAAAGC | hsa-miR-301 hsa- |
| | | miR-301-3p |
| SEQ ID NO: 44 | UAAGUGCUUCCAUGUUUUAGUAG | hsa-miR-302b/hsa- |
| | | miR-302b-3p |
| SEQ ID NO: 45 | UGUAAACAUCCUCGACUGGAAG | hsa-miR-30a-5p |
| SEQ ID NO: 46 | UGUAAACAUCCUACACUCAGCU | hsa-miR-30b/hsa- |
| | | miR-30b-5p |
| SEQ ID NO: 47 | UGUAAACAUCCUACACUCUCAGC | hsa-miR-30c/hsa- |
| | | miR-30c-5p |
| SEQ ID NO: 48 | UGUAAACAUCCCCGACUGGAAG | hsa-miR-30d/hsa- |
| | | miR-30d-5p |
| SEQ ID NO: 49 | UGCUAUGCCAACAUAUUGCCAU | hsa-miR-31*/hsa- |
| | | miR-31-3p |
| SEQ ID NO: 50 | AGGCAAGAUGCUGGCAUAGCU | hsa-miR-31/hsa-miR- |
| | | 31-5p |
| SEQ ID NO: 51 | AAAAGCUGGGUUGAGAGGGCGA | hsa-miR-320/hsa- |
| | | miR-320a-3p |
| SEQ ID NO: 52 | CGCAUCCCCUAGGGCAUUGGUGU | hsa-miR-324-5p |
| SEQ ID NO: 53 | CUGGCCCUCUCUGCCCUUCCGU | hsa-miR-328/hsa- |
| | | miR-328-3p |
| SEQ ID NO: 54 | GCCCCUGGGCCUAUCCUAGAA | hsa-miR-331/hsa- |
| | | miR-331-3p |
| SEQ ID NO: 55 | UCAAGAGCAAUAACGAAAAAUGU | hsa-miR-335/hsa- |
| | | miR-335-5p |
| SEQ ID NO: 56 | GCUGACUCCUAGUCCAGGGCUC | hsa-miR-345/hsa- |
| | | miR-345-5p |
| SEQ ID NO: 57 | CAAUCAGCAAGUAUACUGCCCU | hsa-miR-34*/hsa- |
| | | miR-343-3p |
| SEQ ID NO: 58 | UGGCAGUGUCUUAGCUGGUUGU | hsa-miR-34a/hsa- |
| | | miR-34a-5p |
| SEQ ID NO: 59 | AGGCAGUGUAGUUAGCUGAUUGC | hsa-miR-34c/hsa- |
| | | miR-34c-5p |
| SEQ ID NO: 60 | UUAUCAGAAUCUCCAGGGGUAC | hsa-miR-361/hsa- |
| | | miR-361-5p |
| SEQ ID NO: 61 | UAAUGCCCCUAAAAAUCCUUAU | hsa-miR-365/hsa- |
| | | miR-365a-3p; hsa- |
| | | miR-365b-3p |
| SEQ ID NO: 62 | AAUUGCACUUUAGCAAUGGUGA | hsa-miR-367/hsa- |
| | | miR-367-3p |
| SEQ ID NO: 63 | UUAUAAUACAACCUGAUAAGUG | hsa-miR-374/hsa- |
| | | miR-374a-5p |
| SEQ ID NO: 64 | AUAUAAUACAACCUGCUAAGUG | mmu-miR-374- |
| | | 5p/hsa-miR-374-5p |
| SEQ ID NO: 65 | UAGUGCAAUAUUGCUUAUAGGGU | hsa-miR-454/hsa- |
| | | miR-454-3p |
| SEQ ID NO: 66 | AAGACGGGAGGAAAGAAGGGAG | hsa-miR-483-5p |
| SEQ ID NO: 67 | UCAGGCUCAGUCCCCUCCCGAU | hsa-miR-484 |
| SEQ ID NO: 68 | AGUGGGGAACCCUUCCAUGAGG | mmu-miR-491/hsa- |
| | | miR-491-5p |
| SEQ ID NO: 69 | CAUGCCUUGAGUGUAGGACCGU | hsa-miR-532/hsa- |
| | | miR-532-5p |
| SEQ ID NO: 70 | CCUCCCACACCCAAGGCUUGCA | hsa-miR-532-3p |
| SEQ ID NO: 71 | GAGCUUAUUCAUAAAAGUGCAG | hsa-miR-590-5p |
| SEQ ID NO: 72 | UACCCAUUGCAUAUCGGAGUUG | hsa-miR-660/hsa- |
| | | miR-660-5p |
| SEQ ID NO: 73 | CAACAAAUCACAGUCUGCCAUA | rno-miR-7*/hsa-miR- |
| | | 7-1-3p |
| SEQ ID NO: 74 | AAGGAGCUUACAAUCUAGCUGGG | hsa-miR-708/hsa- |
| | | miR-708-5p |
| SEQ ID NO: 75 | UCUCGCUGGGGCCUCCA | hsa-miR-720 |
| SEQ ID NO: 76 | UGCGGGGCUAGGGCUAACAGCA | hsa-miR-744/hsa- |
| | | miR-744-5p |
| SEQ ID NO: 77 | UAUUGCACUUGUCCCGGCCUGU | hsa-miR-92a/hsa- |
| | | miR-92a-3p |
| SEQ ID NO: 78 | CAAAGUGCUGUUCGUGCAGGUAG | mmu-miR-93/hsa- |
| | | miR-93-5p |
| SEQ ID NO: 79 | CACCCGUAGAACCGACCUUGCG | hsa-miR-99b/hsa- |
| | | miR-99b-5p |
| SEQ ID NO: 80 | UAGCACCAUUUGAAAUCAGUGUU | hsa-miR-29b/hsa- |
| | | miR-29b-3p |
| SEQ ID NO: 81 | CUUUCAGUCGGAUGUUUGCAGC | hsa-miR-30a-3p |
| SEQ ID NO: 82 | CUUUCAGUCGGAUGUUUACAGC | hsa-miR-30e-3p |
| SEQ ID NO: 83 | AAAAGCUGGGUUGAGAGGGCAA | hsa-miR-320B/hsa- |
| | | miR-320b |
| SEQ ID NO: 84 | UAUGUGCCUUUGGACUACAUCG | hsa-miR-455/hsa- |
| | | miR-455-5p |
| SEQ ID NO: 85 | CGCGGGUGCUUACUGACCCUU | hsa-miR-886-3p/hsa- |
| | | miR-886 |
| SEQ ID NO: 86 | CGGGUCGGAGUUAGCUCAAGCGG | hsa-miR-886-5p/hsa- |
| | | miR-886 |
| SEQ ID NO: 87 | AACCCGUAGAUCCGAACUUGUG | hsa-miR-100/hsa- |
| | | miR-100-5p |
| SEQ ID NO: 88 | UCAGUGCAUGACAGAACUUGG | hsa-miR-152/hsa- |
| | | miR-152-3p |
| SEQ ID NO: 89 | CUGUGCGUGUGACAGCGGCUGA | hsa-miR-210/hsa- |
| | | miR-210-3p |
| SEQ ID NO: 90 | UAGCACCAUUUGAAAUCGGUUA | hsa-miR-29c/hsa- |
| | | miR-29c-3p |
| SEQ ID NO: 91 | GCAGUCCAUGGGCAUAUACAC | hsa-miR-455-3p |
| SEQ ID NO: 92 | CACGCUCAUGCACACACCCACA | hsa-miR-574-3p |
| SEQ ID NO: 93 | UAUUCAUUUAUCCCCAGCCUACA | hsa-miR-664/hsa- |
| | | miR-664a-3p |
| SEQ ID NO: 94 | AACCCGUAGAUCCGAUCUUGUG | hsa-miR-99a/hsa- |
| | | miR-99a-5p |
| SEQ ID NO: 95 | UGAGGUAGUAGGUUGUAUAGUU | hsa-let-7a/hsa-let-7a- |
| | | 5p |
| SEQ ID NO: 96 | UGAGGUAGUAGGUUGUAUGGUU | hsa-let-7c/hsa-let-7c- |
| | | 5p |
| SEQ ID NO: 97 | AGAGGUAGUAGGUUGCAUAGUU | hsa-let-7d/hsa-let-7d- |
| | | 5p |
| SEQ ID NO: 98 | UGAGGUAGGAGGUUGUAUAGUU | hsa-let-7e/hsa-let-7e- |
| | | 5p |
| SEQ ID NO: 99 | UGAGGUAGUAGAUUGUAUAGUU | hsa-let-7f/hsa-let-7f- |
| | | 5p |
| SEQ ID NO: 100 | UGAGGUAGUAGUUUGUACAGUU | hsa-let-7g/hsa-let-7g- |
| | | 5p |
| SEQ ID NO: 101 | ACAGAUUCGAUUCUAGGGGAAU | hsa-miR-10b+190/hsa- |
| | | miR-10b-3p |
| SEQ ID NO: 102 | UACCCUGUAGAACCGAAUUUGUG | hsa-miR-10b/hsa- |
| | | miR-10b-5p |
| SEQ ID NO: 103 | UCCCUGAGACCCUUUAACCUGUGA | hsa-miR-125a-5p |
| SEQ ID NO: 104 | UCGGAUCCGUCUGAGCUUGGCU | hsa-miR-127/hsa- |
| | | miR-127-3p |
| SEQ ID NO: 105 | UGUGACUGGUUGACCAGAGGGG | mmu-miR-134/hsa- |
| | | miR-134-5p |
| SEQ ID NO: 106 | CAUCAUCGUCUCAAAUGAGUCU | hsa-miR-136#/hsa- |
| | | miR-136-3p |
| SEQ ID NO: 107 | UUAUUGCUUAAGAAUACGCGUAG | mmu-miR-137/hsa- |
| | | miR-137-3p |
| SEQ ID NO: 108 | UGAGAUGAAGCACUGUAGCUC | hsa-miR-143/hsa- |
| | | miR-143-3p |
| SEQ ID NO: 109 | GUCCAGUUUUCCCAGGAAUCCCU | hsa-miR-145/hsa- |
| | | miR-145-5p |
| SEQ ID NO: 110 | UGAGAACUGAAUUCCAUGGGUU | hsa-miR-146a/hsa- |
| | | miR-146a-5p |
| SEQ ID NO: 111 | UGAGAACUGAAUUCCAUAGGCU | hsa-miR-146b/hsa- |
| | | miR-146b-5p |
| SEQ ID NO: 112 | AAUCAUACACGGUUGACCUAUU | hsa-miR-154#/hsa- |
| | | miR-154-3p |
| SEQ ID NO: 113 | UUAAUGCUAAUCGUGAUAGGGGU | hsa-miR-155/hsa- |
| | | miR-155-5p |
| SEQ ID NO: 114 | AACAUUCAACGCUGUCGGUGAGU | hsa-miR-181a/hsa- |
| | | miR-181a-5p |
| SEQ ID NO: 115 | ACCACUGACCGUUGACUGUACC | hsa-miR-181a-2# |
| | | hsa-miR-181a-2-3p |
| SEQ ID NO: 116 | UAGGUAGUUUCCUGUUGUUGGG | hsa-miR-196b/hsa- |
| | | miR-196b-5p |
| SEQ ID NO: 117 | CCCAGUGUUCAGACUACCUGUUC | hsa-miR-199a/hsa- |
| | | miR-199a-5p |
| SEQ ID NO: 118 | UAACAGUCUCCAGUCACGGCC | hsa-miR-212/hsa- |
| | | miR-212-3p |
| SEQ ID NO: 119 | ACAGCAGGCACAGACAGGCAGU | hsa-miR-214/hsa- |
| | | miR-214-3p |
| SEQ ID NO: 120 | AGUUCUUCAGUGGCAAGCUUUA | hsa-miR-22#/hsa- |
| | | miR-22-5p |
| SEQ ID NO: 121 | AAGCUGCCAGUUGAAGAACUGU | hsa-miR-22/hsa-miR- |
| | | 22-3p |
| SEQ ID NO: 122 | CUCAGUAGCCAGUGUAGAUCCU | hsa-miR-222#/hsa- |
| | | miR-222-5p |
| SEQ ID NO: 123 | ACUGAUUUCUUUUGGUGUUCAG | hsa-miR-29a#hsa- |
| | | miR-29a-5p |
| SEQ ID NO: 124 | CACAUUACACGGUCGACCUCU | hsa-miR-323-3p/hsa- |
| | | miR-323a-3p |
| SEQ ID NO: 125 | ACUGCCCCAGGUGCUGCUGG | hsa-miR-324-3p |
| SEQ ID NO: 126 | UUUUUCAUUAUUGCUCCUGACC | hsa-miR-335#/hsa- |
| | | miR-335-3p |
| SEQ ID NO: 127 | GAACGGCUUCAUACAGGAGUU | hsa-miR-337-5p |
| SEQ ID NO: 128 | UGAGCGCCUCGACGACAGAGCCG | hsa-miR-339-3p |
| SEQ ID NO: 129 | UCCCUGUCCUCCAGGAGCUCACG | hsa-miR-339-5p |
| SEQ ID NO: 130 | GCCUGCUGGGGUGGAACCUGGU | hsa-miR-370/hsa- |
| | | miR-370-3p |
| SEQ ID NO: 131 | AUCAUAGAGGAAAAUCCACGU | hsa-miR-376a/hsa- |
| | | miR-376a-3p |
| SEQ ID NO: 132 | AACAUAGAGGAAAUUCCACGU | hsa-miR-376c/hsa- |
| | | miR-376c-3p |
| SEQ ID NO: 133 | UGGUAGACUAUGGAACGUAGG | mmu-miR-379/hsa- |
| | | miR-379-5p |
| SEQ ID NO: 134 | GAAGUUGUUCGUGGUGGAUUCG | hsa-miR-382/hsa- |
| | | miR-382-5p |
| SEQ ID NO: 135 | AGAUCAGAAGGUGAUUGUGGCU | hsa-miR-383/hsa- |
| | | miR-383-5p |
| SEQ ID NO: 136 | GAAUGUUGCUCGGUGAACCCCU | hsa-miR-409-3p |
| SEQ ID NO: 137 | AAUAUAACACAGAUGGCCUGU | hsa-miR-410/hsa- |
| | | miR-410-3p |
| SEQ ID NO: 138 | UAGUAGACCGUAUAGCGUACG | hsa-miR-411/hsa- |
| | | miR-411-5p |
| SEQ ID NO: 139 | UGUCUUGCAGGCCGUCAUGCA | hsa-miR-431/hsa- |
| | | miR-431-5p |
| SEQ ID NO: 140 | UCUUGGAGUAGGUCAUUGGGUGG | hsa-miR-432/hsa- |
| | | miR-432-5p |
| SEQ ID NO: 141 | AAUCGUACAGGGUCAUCCACUU | hsa-miR-487b/hsa- |
| | | miR-432-5p |
| SEQ ID NO: 142 | UGAAGGUCUACUGUGUGCCAGG | hsa-miR-493/hsa- |
| | | miR-493-3p |
| SEQ ID NO: 143 | UGAAACAUACACGGGAAACCUC | hsa-miR-494/hsa- |
| | | miR-494-3p |
| SEQ ID NO: 144 | AAACAAACAUGGUGCACUUCUU | mmu-miR-495/hsa- |
| | | miR-495-3p |
| SEQ ID NO: 145 | GGAGAAAUUAUCCUUGGUGUGU | hsa-miR-539/hsa- |
| | | miR-539-5p |
| SEQ ID NO: 146 | AAACAUUCGCGGUGCACUUCUU | hsa-miR-543 |
| SEQ ID NO: 147 | ACAGUCUGCUGAGGUUGGAGC | hsa-miR-622 |
| SEQ ID NO: 148 | UGGUGGGCCGCAGAACAUGUGC | hsa-miR-654/hsa- |
| | | miR-654-5p |
| SEQ ID NO: 149 | UUUGUGACCUGGUCCACUAACC | hsa-miR-758/hsa- |
| | | miR-758-3p |
| SEQ ID NO: 150 | UUAAUAUCGGACAACCAUUGU | hsa-miR-889/hsa- |
| | | miR-889-3p |
| SEQ ID NO: 151 | UCGUACCGUGAGUAAUAAUGCG | hsa-miR-126/hsa- |
| | | miR-126-3p |
| SEQ ID NO: 152 | UCACAGUGAACCGGUCUCUUU | hsa-miR-128a/hsa- |
| | | miR-128-3p |
| SEQ ID NO: 153 | UUUGGUCCCCUUCAACCAGCUG | hsa-miR-133a/hsa- |
| | | miR-133a-3p |
| SEQ ID NO: 154 | UAUGGCUUUUUAUUCCUAUGUGA | hsa-miR-135a/hsa- |
| | | miR-135a-5p |
| SEQ ID NO: 155 | UAUGGCUUUUCAUUCCUAUGUGA | hsa-miR-135b/hsa- |
| | | miR-135b-5p |
| SEQ ID NO: 156 | UAACACUGUCUGGUAAAGAUGG | hsa-miR-141/hsa- |
| | | miR-141-3p |
| SEQ ID NO: 157 | UCAGUGCACUACAGAACUUUGU | hsa-miR-148a/hsa- |
| | | miR-148a-3p |
| SEQ ID NO: 158 | GUGAAUUACCGAAGGGCCAUAA | hsa-miR-183#/hsa- |
| | | miR-183-3p |
| SEQ ID NO: 159 | UCGUGUCUUGUGUUGCAGCCGG | mmu-miR-187/hsa- |
| | | miR-187-3p |
| SEQ ID NO: 160 | CUGACCUAUGAAUUGACAGCC | hsa-miR-192/hsa- |
| | | miR-192-5p |
| SEQ ID NO: 161 | UGUAACAGCAACUCCAUGUGGA | hsa-miR-194/hsa- |
| | | miR-194-5p |
| SEQ ID NO: 162 | UAAUACUGCCUGGUAAUGAUGA | hsa-miR-200b/hsa- |
| | | miR-200b-3p |
| SEQ ID NO: 163 | UAAUACUGCCGGGUAAUGAUGGA | hsa-miR-200c/hsa- |
| | | miR-200c-3p |
| SEQ ID NO: 164 | GUGAAAUGUUUAGGACCACUAG | hsa-miR-203/hsa- |
| | | miR-203a-3p |
| SEQ ID NO: 165 | UCCUUCAUUCCACCGGAGUCUG | hsa-miR-205/hsa- |
| | | miR-205-5p |
| SEQ ID NO: 166 | CAAAGUGCUCAUAGUGCAGGUAG | hsa-miR-20b/hsa- |
| | | miR-20b-5p |
| SEQ ID NO: 167 | CAGUGCAAUGAUAUUGUCAAAGC | hsa-miR-301b/hsa- |
| | | miR-301b-3p |
| SEQ ID NO: 168 | ACUUAAACGUGGAUGUACUUGCU | hsa-miR-302a#/hsa- |
| | | miR-302a-5p |
| SEQ ID NO: 169 | UAAGUGCUUCCAUGUUUUGGUGA | hsa-miR-302a/hsa- |
| | | miR-302a-3p |
| SEQ ID NO: 170 | ACUUUAACAUGGAAGUGCUUUC | hsa-miR-302b#/hsa- |
| | | miR-302b-5p |
| SEQ ID NO: 171 | UUUAACAUGGGGGUACCUGCUG | hsa-miR-302c+190/hsa- |
| | | miR-302c-5p |
| SEQ ID NO: 172 | UAAGUGCUUCCAUGUUUCAGUGG | hsa-miR-302c/hsa- |
| | | miR-302c-3p |
| SEQ ID NO: 173 | ACUUUAACAUGGAGGCACUUGC | hsa-miR-302d#/hsa- |
| | | miR-302d-5p |
| SEQ ID NO: 174 | UAAGUGCUUCCAUGUUUGAGUGU | hsa-miR-302d/hsa- |
| | | miR-302d-3p |
| SEQ ID NO: 175 | UCCGUCUCAGUUACUUUAUAGC | hsa-miR-340#/hsa- |
| | | miR-340-3p |
| SEQ ID NO: 176 | UUAUAAAGCAAUGAGACUGAUU | hsa-miR-340/hsa- |
| | | miR-340-5p |
| SEQ ID NO: 177 | CAAUCACUAACUCCACUGCCAU | hsa-miR-34b/hsa- |
| | | miR-34b-3p |
| SEQ ID NO: 178 | AAUUGCACGGUAUCCAUCUGUA | hsa-miR-363/hsa- |
| | | miR-363-3p |
| SEQ ID NO: 179 | AAAGUGCUGCGACAUUUGAGCGU | hsa-miR-372/hsa- |
| | | miR-372-3p |
| SEQ ID NO: 180 | UGAGGGGCAGAGAGCGAGACUUU | hsa-miR-423-5p |
| SEQ ID NO: 181 | GUGACAUCACAUAUACGGCAGC | hsa-miR-489/hsa- |
| | | miR-489-3p |
| SEQ ID NO: 182 | AAGUGCUGUCAUAGCUGAGGUC | hsa-miR-512-3p |
| SEQ ID NO: 183 | UUCUCCAAAAGAAAGCACUUUCUG | hsa-miR-515-5p |
| SEQ ID NO: 184 | AAAGUGCUUCCUUUUAGAGGG | hsa-miR-520b/hsa- |
| | | miR-520b-3p |
| SEQ ID NO: 185 | AAAGUGCUUCCUUUUAGAGGGU | hsa-miR-520c-3p |
| SEQ ID NO: 186 | GUCCGCUCGGCGGUGGCCCA | hsa-miR-572 |
| SEQ ID NO: 187 | UGUGUCACUCGAUGACCACUGU | hsa-miR-597/hsa- |
| | | miR-597-5p |
| SEQ ID NO: 188 | GUUCUCCCAACGUAAGCCCAGC | hsa-miR-629/hsa- |
| | | miR-629-3p |
| SEQ ID NO: 189 | AGGGAUCGCGGGCGGGUGGCGGCCU | hsa-miR-638 |
| SEQ ID NO: 190 | UGGAAGACUAGUGAUUUUGUUGU | dme-miR-7/hsa-miR- |
| | | 7-5p |
| SEQ ID NO: 191 | ACUCCAGCCCCACAGCCUCAGC | hsa-miR-766/hsa- |
| | | miR-766-3p |
| SEQ ID NO: 192 | UGAGACCUCUGGGUUCUGAGCU | hsa-miR-769-5p |
| SEQ ID NO: 193 | UCCAUUACACUACCCUGCCUCU | hsa-miR-885-5p |
| SEQ ID NO: 194 | AUAAAGCUAGAUAACCGAAAGU | hsa-miR-9#/hsa-miR- |
| | | 9-3p |
| SEQ ID NO: 195 | UCUUUGGUUAUCUAGCUGUAUGA | hsa-miR-9/hsa-miR- |
| | | 9-5p |
| SEQ ID NO: 196 | ACUGCUGAGCUAGCACUUCCCG | hsa-miR-93#/hsa- |
| | | miR-93-3p |
| SEQ ID NO: 197 | UCUUCUCUGUUUUGGCCAUGUG | hsa-miR-942/hsa- |
| | | miR-942-5p |
| SEQ ID NO: 198 | UUCAACGGGUAUUUAUUGAGCA | hsa-miR-95/hsa-miR- |
| | | 95-3p |
| SEQ ID NO: 199 | AGCAGCAUUGUACAGGGCUAUCA | hsa-miR-107 |
| SEQ ID NO: 200 | UUUCCGGCUCGCGUGGGUGUGU | hsa-miR-1180-3p |
| SEQ ID NO: 201 | UAAGGCACGCGGUGAAUGCC | mmu-miR-124a/hsa- |
| | | miR-124-3p |
| SEQ ID NO: 202 | ACUGCAUUAUGAGCACUUAAAG | hsa-miR-20a#/hsa- |
| | | miR-20a-3p |
| SEQ ID NO: 203 | UGUCAGUUUGUCAAAUACCCCA | hsa-miR-223-3p |
| SEQ ID NO: 204 | GAAGUGCUUCGAUUUUGGGGUGU | hsa-miR-373-3p |
| SEQ ID NO: 205 | ACUGGACUUGGAGUCAGAAGG | hsa-miR-378/hsa- |
| | | miR-378-3p |
| SEQ ID NO: 206 | CGUCAACACUUGCUGGUUUCCU | hsa-miR-505-3p |
| SEQ ID NO: 207 | CACUCAGCCUUGAGGGCACUUUC | hsa-miR-512-5p |
| SEQ ID NO: 208 | CAAAGUGCCUCCCUUUAGAGUG | hsa-miR-519d-3p |
| SEQ ID NO: 209 | CUCUUGAGGGAAGCACUUUCUGU | hsa-miR-526b/hsa- |
| | | miR-526b-5p |
| SEQ ID NO: 210 | UGGUCUAGGAUUGUUGGAGGAG | hsa-miR-601 |
| SEQ ID NO: 211 | GACUAUAGAACUUUCCCCCUCA | hsa-miR-625+190/hsa- |
| | | miR-625-3p |
| SEQ ID NO: 212 | UGUGCUUGCUCGUCCCGCCCGCA | hsa-miR-636 |
| SEQ ID NO: 213 | UCUAGGCUGGUACUGCUGA | hsa-miR-645 |
| SEQ ID NO: 214 | CUGCCCUGGCCCGAGGGACCGA | hsa-miR-874/hsa- |
| | | miR-874-3p |

### EXAMPLE I

All the experiments done by using cells were performed in accordance with the current Italian law and approved by the Ethics Committee.

Samples from isolated human donors were collected and used under written informed consents according to the requirements of national patent law.

Isolation of Multipotent Mesenchymal Stem Cells from Cord Blood

Multipotent Mesenchymal Stem Cells (MSCs) from cord blood (CB) were isolated from different samples of CB (n=96 for the standard protocol, n=23 for the optimized protocol) by processing CB centrifuging it at 500 xg for 15 minutes and harvesting the buffy coat containing white blood cells. Next, the buffy coat was incubated for 20 minutes at room temperature (RT) with 50 μL/mL of RosetteSep hematopoietic cell-immunodepleting MSC-enriching antibody cocktail (StemCell technologies). Then, the mixture was diluted 1:3 with PBS, 2 mM EDTA, 0.5% HSA and loaded 1:2 onto 1.077 g/mL Ficoll for density gradient separation performed at 200 xg for 25 minutes. Next, mononucleated cells were harvested at the interface with Ficoll and seeded at 1×10⁶ cells/cm². For the standard protocol aMEM supplemented with 20% FBS was preferably used as culture medium, and the cells were preferably seeded at 4,000 cells/cm² for replating. For the optimized protocol SPE-IV medium supplemented with 20% FBS and 2 mM L-glutamine was preferably used as culture medium, and the cells were preferably seeded at 1,500 cells/cm² for replating.

With the aim of facilitating the assessment of colony formation within the context of CB primary stromal cell culture, characterized by a mixed cell environment, only colonies composed of at least 10 CB-derived fibroblastic-like cells and/or colonies of at least 1 mm in diameter were considered successfully processed.

The adherent and non-adherent “contaminant” cell types were removed by negative immunodepletion of T lymphocytes by CD3, monocytes by CD14, B lymphocytes by CD19, red blood cells by glycophorin A and granulocytes by CD66b from CB mononuclear cells before seeding.

The adherent contaminant cells were removed after MSC colony formation during the first trypsinization, as adherent contaminant cells remained attached to the culture surface, while the treatment detached MSC.

Concerning the optimized isolation protocol, the purified cells were preferably cultured onto standard tissue culture-treated plastic surfaces, preferably without additional coating. Initial seeding for the isolation protocol was preferably 1×10⁶ cells/cm². The culture medium was preferably SPE-IV medium (ABCell-Bio) supplemented with 20% FBS and 2 mM L-glutamine, and the cells were preferably seeded at 1,500 cells/cm² for replating.

Under these conditions, positive events were observed in almost 80% of the processed samples (23 out of 29). Following this isolation procedure, the presence of “contaminant” adherent cell types were dramatically decreased.

The established CBMSC populations showed enhanced growth properties, with regard to both CPD peak values and CFU-F assay so we named these cells Long (LL)-CBMSCs.

They were positive for canonical MSC markers CD90, CD105 and CD73, while they were negative for CD45 and CD34.

Moreover, they express NG2 and CD56, but almost no expression of CD271.

Telomere length was also assessed in spite of a dramatic and significant decrease in mean telomere length comparing passage 0 to passage 1 CBMSCs, this parameter was subsequently maintained at a constant length.

LL-CBMSCs showed a high CPD value—CDP>30—and they were capable to generate many secondary colonies under low density-seeding conditions (colony-forming unit-fibroblasts assay).

LL-CBMSCs grew in adherence to plastic surfaces, with a fibroblast-like morphology, and they showed a typical MSC immunophenotype. Moreover, their differentiation potential into mesodermal derivatives was also investigated as requested by the ISCT MSC definition criteria (doi: 10.1080/14653240600855905).

Induced Cellular Reprogramming

One major drawback of LL-CBMSCs as extracellular vescicle (EV) source is that, unlike pluripotent stem cells, they are primary cells with limited lifespan in vitro. Even though LL-CBMSCs can be extensively expanded thanks to their growth properties, an immortalized cell line would have the advantage of an unlimited use. However, the preservation of EV therapeutic features after immortalization has to be still demonstrated.

LL-CBMSCs reprogramming was achieved using a non-integrating viral-based system, which guarantees the expression of the following reprogramming factors: OCT4, SOX2, KLF4, cMYC.

Reprogramming System

LL-CBMSCs reprogramming was achieved using CytoTune-iPS 2.0 Reprogramming System vectors (Thermo Fisher Scientific, Waltham, Mass., USA) that are based on the genome of a non-transmissible form of Sendai virus (SeV) to deliver and to express the genetic factors (OCT4, SOX2, KLF4, cMYC) necessary to reprogram somatic cells into human induced pluripotent stem cells (iPSCs). Three different vectors express the reprogramming factors: one contains the KLF4, OCT4 and SOX2 sequences (KOS vector); one contains only KLF4 sequence (K vector); the last contains only cMYC sequence (M vector). These vectors are non-integrating and they remain in the cytoplasm, meaning they are zero-footprint. An important modification introduced in the SeV genome is that the gene encoding for the F protein, essential for the viral envelope to fuse with cell membranes, is deleted from all reprogramming vectors.

Therefore, they are unable to produce infectious particles from infected cells.

Transduction Feasibility

The feasibility of LL-CBMSCs infection and forced exogenous protein expression was investigated. For this purpose, an emerald green fluorescent protein (emGFP)-expressing variant of this system was used and the results showed that LL-CBMSCs were successfully infected and the protein expressed.

Reprogramming Protocol

The protocol applied to reprogram LL-CBMSCs is the following:
1) On day-2, LL-CBMSCs were plated into two wells of a 6-well plate in LL-CBMSC standard medium at two different cell densities (150,000 and 200,000 cells/cm²);
2) On day 0, LL-CBMSCs of the well showing 70-80% confluent were transduced using the CytoTune-iPS 2.0 Sendai reprogramming vectors at MOI=5 (KOS and M vectors) and MOI=3 (K vector), following the procedure previously described;
3) LL-CBMSCs were then incubated overnight at 37° C., 5% CO₂;
4) On day 1, the medium was replaced with fresh medium to remove viral particles;
5) On day 2-6, the medium was replaced every other day;
6) On day 7, the transduced cells were detached and seeded on matrigel (BD)-coated culture Petri dishes in LL-CBMSC medium;
7) On day 8, the LL-CBMSC medium was switched to StemMACS iPS-Brew XF medium (Miltenyi Biotec, Bergisch Gladbach, Germany);
8) On day 9-28, the medium was replaced every day and the culture vessels were monitored for the emergence of human iPSC (hiPSC) colonies;
9) When hiPSC colonies were ready for transfer, manual mechanical picking using a 150 μm diameter needle mounted on a stripper micropipette was performed. Colony clumps of 20-200 cells were seeded in StemMACS medium onto matrigel-coated culture Petri dishes for expansion.

hiPSCs were cultured as Human embryonic stem cells (hESC) in StemMACS iPS-Brew XF medium (Miltenyi Biotec—MB) onto matrigel (BD)-coated culture dishes. The medium was changed every day. At 80% confluence, the colonies were detached by ethylenediaminetetraacetic acid (EDTA;—Thermo Fisher Scientific) passaging. Briefly, the medium was removed and the cells washed with PBS (Thermo Fisher Scientific). Then, the cells were incubated with 5 mM EDTA for 4 minutes in a 37° C., 5% CO2 incubator. EDTA was gently removed and the colonies detached and disgregated in 20-200 cell clumps by pipetting, using StemMACS iPS-Brew medium (MB). The cells were seeded in new matrigel-coated culture dishes, observing a 1:3-1:8 split ratio corresponding to a cell density concentration of 30,000-10,000 cells/cm², depending on experimental needs. Freezing medium was composed of StemMACS iPS-Brew XF medium (MB) supplemented with 10% DMSO (Bioniche Pharma, Costelloe, Ireland); thawing medium was composed of StemMACS iPS-Brew XF medium (MB) supplemented with 10 μM Rock inhibitor (Stemcell technologies). The day after passaging and thawing, the medium was not changed.

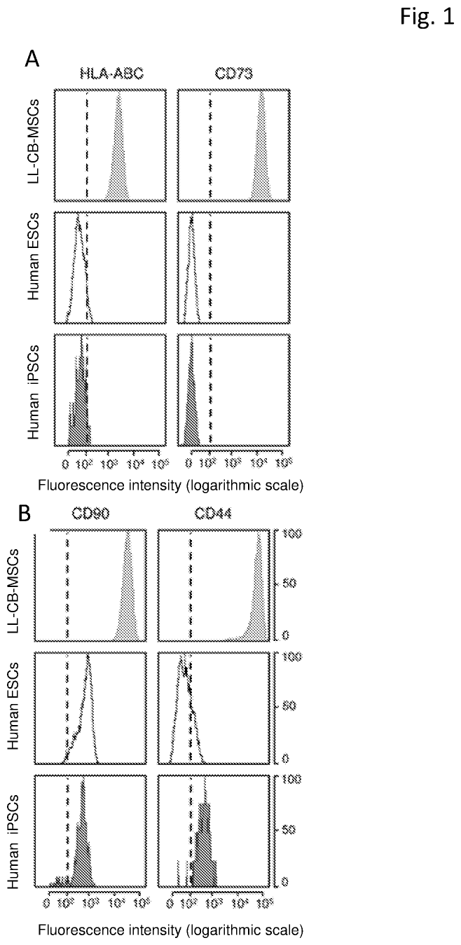

The results showed that cell morphology changes were not visible the first day, whereas small colonies composed of highly packed cells with epithelial-like shape were detected starting one week after. After LL-CBMSC-derived bona fide hiPSC colonies (referred to as hiPSCs from now on) reached confluence at week 3 post-infection, hiPSC lines (n=3) were generated by manual mechanical “picking” of single colonies, using a 150 μm diameter needle mounted on a stripper micropipette. The established hiPSC lines grew for more than 40 passages and showed a typical pluripotent stem cell morphology, growing as densely packed epithelial-like cells forming colonies with well-defined edges. In order to assess the immunophenotypic status of hiPSCs, the expression of classic MSC markers was addressed, by flow cytometry. Noteworthy, human leukocyte antigens A, B and C (HLA-ABC) expression on hiPSC membrane surface was drastically reduced, compared to parental LL-CBMSCs (FIG. 1A), in line with a pluripotent stem cell phenotype. Analogous to HLA-ABC, CD73 expression was completely lost in hiPSCs (FIG. 1A). On the contrary, CD90 and CD44 were retained, even though the signal intensity was strongly reduced (FIG. 1 B).

Therefore, HLA-ABC and CD73 represent a minimal immunophenotype panel that could be useful to assess the successful reprogramming of MSCs.

Finally, to ensure the maintenance of genomic stability, the karyotype of parental and reprogrammed cells was investigated by using a standard protocol. Importantly, no chromosomal abnormalities were detected.

Stemness and Pluripotency Assessment

The hiPSC lines grow for many passages (>40), and they were thoroughly characterized to assess their identity.

Alkaline Phosphatase Activity

Alkaline phosphatase (AP) activity was assessed, as a typical feature of pluripotent stem cells. Direct alkaline phosphase activity was assessed using the Alkaline Phosphatase (AP) Live Stain kit (Life Technologies, Carlsbad, Calif. United States). The analysis was performed on hiPSC and hESC colonies (positive control) and on LL-CBMSCs (negative control). All cells were grown on chamber slides (BD), following the cell culture conditions previously described.

The following steps were performed:
The medium was removed, and the cells were washed with PBS;
The cells were incubated for 15 minutes at room temperature (RT) with a 4% paraformaldehyde (PFA—Thermo Fisher Scientific) fixative solution (PBS-diluted);
The fixative solution was removed and the cells were washed with PBS;
The cells were incubated 30 minutes at RT with 1 μL of AP Live Stain solution in 0.5 mL of DMEM/F-12 (Sigma-Aldrich, Saint Louis, Mo., USA);
The AP Live Stain solution was removed and the cells were washed twice with DMEM/F-12 to reduce background signal.

The detection of fluorescent colonies was performed on an inverted phase-contrast microscope (Nikon Eclipse Ti)

Notwithstanding, also parental non-reprogrammed LL-CBMSCs showed strong AP activity, suggesting that it is not a useful parameter to consider in the context of MSC reprogramming.

Pluripotency Network-Related Genes

The expression of key pluripotency network-related genes was investigated by Real Time qRT-PCR, compared to parental LL-CBMSCs as negative control, and to a pluripotent stem cell line as positive control. The RNeasy Plus Mini kit (Qiagen) was used to extract RNA from cell pellets previously frozen at −20° C. The samples were first lysed and homogenized in RNeasy Plus lysis buffer (Qiagen). Then, the lysate was filtered through a gDNA-eliminator spin column (Qiagen), which allows efficient removal of genomic DNA after a centrifugation at ≥8000 xg for 30 seconds. Next, the flow-through was collected and 350 μL of 70% ethanol were added. The samples were then transferred to an RNeasy spin column (Qiagen), and centrifuged for 15 seconds at 800 xg. Then, 700 μL of Buffer RW1 (Qiagen) were added to the RNeasy spin column and centrifuged again for 15 seconds at ≥8000 xg to wash the spin column membrane. The flow-through was discarded. Then, 500 μL RPE Buffer were added to the RNeasy spin column and centrifuged for 15 seconds at ≥8000 xg to wash again the spin column membrane. This passage was repeated one more time with a 2 minute centrifugation. RNA was then eluted in 30 μL of distilled water. RNA quantification was performed with a Nanodrop 1000 instrument, reading absorbance at 260 nm; purity was assessed by A260/A230 (organic compound contamination) and A260/A280 (protein contamination) ratios. Integrity of GelRed (Biotium)-stained RNA was assessed by 1% agarose gel electrophoresis at 100 V (1 hour), after staining detection using the GelDoc XR instrument (Bio-Rad). Each RNA sample (800 ng) was retrotranscribed with the iScript cDNA synthesis kit (Bio-Rad). Briefly, 4 μL of 5× iScript reaction mix (salts, dNTPs, oligo(dT), examer random primers) and 1 μL of iScript reverse transcriptase were added to 15 μL of RNA in a PCR tube. Then, the tubes were placed in a thermal cycler (DNA Engine Peltier Thermal Cycler PTC-200; Bio-Rad) and the following steps were performed: 25° C. for 5 minutes, 42° C. for 30 minutes, 85° C. for 5 minutes and 4° C. until storage. To assess gene expression, Real Time qRT-PCR was performed. A 10 μL PCR reaction mix was prepared with 5 μL of 2× SsoFast EvaGreen Supermix (Bio-Rad), 500 nM of forward and reverse primers and 12 ng of cDNA. Then, the reaction mix for each sample was loaded into a Real Time qRT-PCR 96-well plate, in triplicate. Amplification data were studied using the Bio-Rad CFX Manager software (Bio-Rad); technical replicate values were checked for differences inferior to 0.5 threshold cycles (Ct). The melting curve relative to every reaction was examined to exclude aspecific amplification. In addition, amplification reactions with Ct values higher than 35 were not considered. Finally, gene expression was calculated with the ΔΔCt method, using GAPDH as house-keeping gene and normalizing to hESC (positive control). When feasible, the primers were designed to anneal to exon-exon junction regions of the mature transcripts and to amplify exons separated by at least one intron, to decrease the risk of unspecific amplification of genomic DNA. PCR cycling conditions and primer sequences are provided in Tables 2 and 3.

TABLE 2
PCR cycling conditions

| | Temperature | Time | Cycles |
|---|---|---|---|
| Initial denaturation | 98° C. | 30 seconds | 1 |
| Denaturation | 95° C. | 5 seconds | |
| Annealing and | 60° C. | 20 seconds | 46 |
| Polymerization | | | |
| Melting curve | 65° C. | 5 seconds | 1 |
| | 95° C. | | |
| Hold | 4° C. | >5 minutes | 1 |

TABLE 3
Primer sequences

| | Name | Sequences |
|---|---|---|
| eepi | GAPDH | forward primer: CCCTTCATTGACCTCAACTACATG |
| | | reverse primer: TGGGATTTCCATTGATGACAAGC |
| Target | KLF4 | forward primer: ACGATCGTGGCCCCGGAAAAGGACC |
| genes | | reverse primer: TGATTGTAGTGCTTTCTGGCTGGGCTCC |
| | LIN28A | forward primer: TTCGGGTTCCAGGGAGAC |
| | | reverse primer: CGTTTCAACACAGTAAGGTAGG |
| | cMYC | forward primer: GCGTCCTGGGAAGGGAGATCCGGAGC |
| | | reverse primer: TTGAGGGGCATCGTCGCGGGAGGCTG |
| | NANOG | forward primer: GAGATGCCTCACACGGAGACTG |
| | | reverse primer: GGGTTGTTTGCCTTTGGGACTG |
| | OCT4A | forward primer: CAGCCTGAGGGCGAAGCAG |
| | | reverse primer: GCTTTGATGTCCTGGGACTCCTC |
| | SOX2 | forward primer: GTACTGGCGAACCATTCTTG |
| | | reverse primer: AAATTACCAACGGTGTCAACCTG |

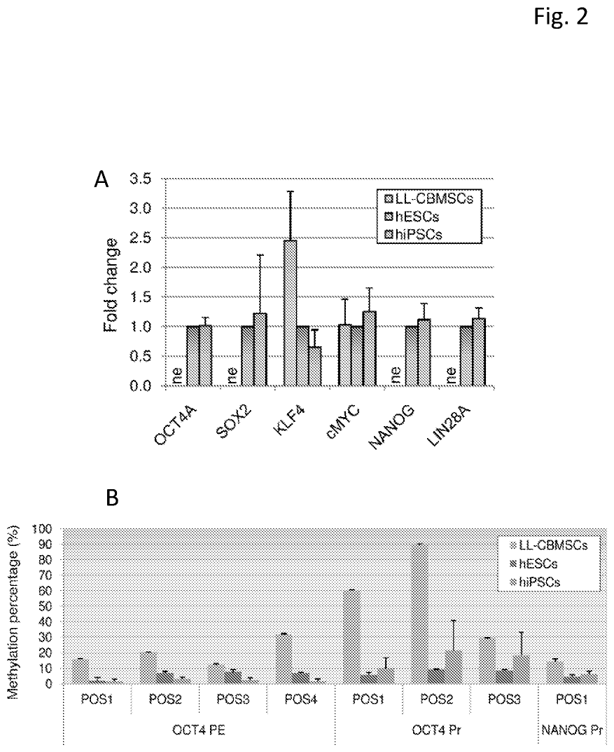

The results are reported in FIG. 2 and they demonstrate that established hiPSC lines are characterized by an increased gene expression for OCT4 (absent in parental cells) and SOX2 (absent in parental cells), two well-known pluripotency markers. KLF4 was more expressed in parental cells (5-fold change), while cMYC was expressed at similar levels. More importantly, also NANOG (absent in parental cells) and LIN28A (absent in parental cells), not present in the cocktail of exogenously-encoded reprogramming factors, showed a gene expression similar to that of the pluripotent control. This clearly indicates that the expression of exclusive pluripotency-associated genes was switched on at the endogenous level. To detect expression of pluripotency-associated markers at the protein level, immunofluorescence analyses were performed on undifferentiated hiPSC colonies. Both OCT4/SSEA4 and SOX2/TRA1-60 double staining revealed strong protein expression.

Epigenetic Profile of hiPSCs

The epigenetic state of crucial gene regulatory regions was addressed on hiPSC bisulfite-treated DNA by pyrosequencing compared to LL-CBMSCs as negative control, and to a pluripotent stem cell line as positive control.

Cell pellets were collected from subconfluent LL-CBMSC, hESC H9 or hiPSC cultures and immediately frozen at -80° C. until use. DNA was extracted by the QIAamp DNA Blood Mini Kit (51104; Qiagen). Briefly, 200 μL of buffer AL were added to the sample and mixed by pulse-vortexing for 15 seconds. Then, the samples were incubated at 56° C. for 10 minutes, after which 200 μL of ethanol (96-100%) were added to the sample and mixed again by pulse-vortexing for 15 seconds. The mixture was applied to the QIAamp Mini spin columns and centrifuged at 6000 xg for 1 minute, after which the filtrate was discarded. Then, 500 μL of buffer AW1 were added to the QIAamp Mini spin columns and centrifuged at 6000 x g for 1 minute, after which the filtrate was discarded. The same procedure was repeated for buffer AW2, but at full speed (20,000 xg) for 3 minutes. To elute the filter-bound DNA, 200 μL of buffer AE were added to the QIAamp Mini spin columns, which were incubated at room temperature for 1 minute, before they were centrifuged at 6000 x g for 1 minute. DNA quantification was performed with a Nanodrop 1000 instrument, reading absorbance (A) at 260 nm; purity was assessed by A260/A230 (organic compound contamination) and A260/A280 (protein contamination) ratios; integrity was assessed by agarose gel electrophoresis, as previously described.

Each sample DNA (500 ng) was treated with the EZ DNA Methylation-Gold Kit (Zymo Research) to obtain bisulfite conversion. Briefly, 130 μL of the CT conversion reagent were added to 20 μL of DNA in a PCR tube. Then, the tube was placed in a thermal cycler and the following steps were performed: 98° C. for 10 minutes, 64° C. for 2.5 hours and 4° C. for storage. Then, 600 μL of M-binding buffer were added to a Zymo-Spin IC column, which was placed into a collection tube. The sample was loaded into the column and it was mixed by inverting the column several times. Centrifugation at full speed (>10,000 x g) for 30 seconds was performed and the flow-through discarded. Then, 100 μL of M-wash buffer were added to the column and centrifugation at full speed for 30 seconds was performed. Next, 200 μL of M-desulphonation buffer were added to the column, which was let stand at room temperature for 20 minutes. After the incubation, centrifugation at full speed for 30 seconds was performed, followed by two washing steps with 200 μL of M-wash buffer. The column was placed into a 1.5 mL microcentrifuge tube. Finally, bisulfite-treated DNA was eluted in 30 μL of M-elution buffer and stored at −20° C. for later use.

To analyze DNA methylation, a 50 μL PCR reaction was carried out with 25 μL of GoTaq Hot Start Green Master mix (Promega), 10 μM of forward primer, 10 μM of biotinylated reverse primer and 500 ng of bisulfite-treated DNA. PCR cycling conditions and primer sequences are provided in table 1 and 2, respectively. Biotin-labeled primers were used to purify the final PCR product with sepharose beads: 10 μL of PCR product were bound to 1 μL of Streptavidin Sepharose HP affinity chromatography medium (Amersham Biosciences) in presence of 40 μL of binding buffer (Amersham Biosciences), after a 10 minute incubation in agitation. Sepharose beads containing the immobilized PCR product were purified, washed, denatured with 0.2 M NaOH and washed again with the Pyrosequencing Vacuum Prep Tool (Pyrosequencing, Inc.), according to the manufacturer's instructions. Pyrosequencing primer (0.3 μM) was annealed to the purified single-stranded PCR product in presence of 15 μL of annealing buffer, during an incubation of 2 minutes at 85° C., then pyrosequencing was performed in duplicate with the PyroMark MD System (Pyrosequencing, Inc.). The percentage of methylated cytosines was calculated as the number of methylated cytosines divided by the sum of methylated and unmethylated cytosines, multiplied by 100%.

TABLE 4
PCR cycling conditions

| Temperature | Time | Cycles |
|---|---|---|
| 95° C. | 5 minutes | 1 |
| 95° C. | 30 seconds | 45 |
| 54° C. | 45 seconds | |
| for NANOG promoter | | |
| 59° C. | | |
| for OCT4 promoter | | |
| 56° C. | | |
| for OCT4 proximal enhancer | | |
| 72° C. | 45 seconds | |
| 72° C. | 5 minutes | 1 |
| 6° C. | For ever | 1 |

TABLE 5
Primer sequences

| Assay | Sequences |
|---|---|
| NANOG | Forward TTGTTGTTGGGTTTGTTTTTAGG (SEQ ID NO: 215) |
| promoter | Reverse BIO-ACCTTAAACCCACCCCTCCTA (SEQ ID NO: 216) |
| | Sequencing TTTGGTGAGATTGGTAGA (SEQ ID NO: 217) |
| OCT4 | Forward AAGTTTTTGTGGGGGATTTGTAT (SEQ ID NO: 218) |
| promoter | Reverse BIO-CCACCCACTAACCTTAACCTCTA (SEQ ID NO: |
| | 219) |
| | Sequencing 1 TGAGGTTTTGGAGGG (SEQ ID NO: 220) |
| | Sequencing 2 GTTATTATTATTAGGTAAATATTTT (SEQ ID NO: |
| | 221) |
| OCT4 | Forward AGTTTTAGGATATTTAGGTTAGGTTTAGAA (SEQ ID |
| proximal | NO: 222) |
| enhancer | Reverse BIO-CAACAACCCCCCTCTACAAT (SEQ ID NO: 223) |
| | Sequencing 1 GGATATTTAGGTTAGGTTTAGAAA (SEQ ID NO: |
| | 224) |
| | Sequencing 2 GTATATTTTTTAATTTGTTAGGTT (SEQ ID NO: |
| | 225) |

The results are summarized in FIG. 2B and showed complete methylation remodeling for CpG islands in various positions along OCT4 promoter and proximal enhancer sequences. The same was found for NANOG promoter, further confirming fully acquisition of pluripotent features for LL-CBMSCs following reprogramming.

hiPSCs Differentiation

Human iPSC differentiation properties were investigated to complete the characterization of the cell lines generated. Protein marker expression for derivatives of the three germ layers was assessed by immunofluorescence, after in vitro differentiation protocols were applied.

To obtain endoderm and mesoderm cell derivatives, an initial embryoid body (EB) formation step was performed. Briefly, hiPSC and hESC colonies were detached using the previously described non-enzymatic method. Next, cell aggregation was promoted by transferring 150 μL of cell suspension per well into a low-attachment V-bottom 96-well plate. The cells were cultured in KO-DMEM (Sigma-Aldrich) supplemented with 20% KO-Serum Replacement (KO-SR; Life Technologies), 2 mM Glutamax (Life Technologies), 50 μM 2-mercaptoethanol (Life Technologies), 10 mL/L penicillin/streptomycin (Sigma-Aldrich) and 1mM non-essential aminoacids (Life Technologies) (KO medium) for 3-4 days to allow EB formation. Then, EBs were transferred into low-attachment 24-well plate and maintained in suspension in KO medium for 2-3 supplementary days. Finally, the EBs were transferred onto 0.1% gelatin (StemCell Technologies)-coated glass chamber slides (BD). Endodermal differentiation medium was composed of DMEM (Sigma Aldrich), 20% FBS (Life Technologies), 2 mM L-glutamine (Capricorn Scientific), 0.1 mM 2-mercaptoethanol (Life Technologies), 1 mM non-essential aminoacids (Life Technologies) and 10 mL/L penicillin/streptomycin (Sigma-Aldrich). The mesodermal differentiation medium was the same used for endodermal differentiation, supplemented with 100 mM ascorbic acid (Sigma-Aldrich). The EBs were maintained onto the gelatin-coated glass chamber slides for 2-3 weeks. The medium was replaced twice a week.

In order to obtain differentiation to early neuro-epithelial cells, Neurobasal (Life Technologies), 1X N2 (Life Technologies), 1X B27 (Life Technologies), 10 mL/L penicillin/streptomycin (Sigma-Aldrich), 2 mM L-glutamine (Capricorn Scientific) (N2-B27 medium) was used. The following protocol was carried out:
hESCs and hiPSCs were grown in chamber slides (BD), in the previously described culture conditions;
At 80% confluence, the medium was removed and KO medium was added to the cells;
The KO medium was not changed for 5 days;
At day 5, the medium was carefully aspirated and replaced entirely with 75% KO medium, 25% N2-B27 medium;
At day 7, the medium was changed to 50% KO medium, 50% N2-B27 medium;
At day 9, the medium was changed to 25% KO medium, 75% N2-B27 medium;
At day 12, the medium was carefully aspirated and replaced entirely with 100% N2-B27 medium;
Between day 12 and day 15, the medium was partially changed once: ⅓ of medium was kept and ⅔ of fresh N2-B27 medium was added;
When the cells acquired an early neuro-epithelial-like morphology, the medium was removed and the samples were processed for immunofluorescence.

The results showed that hiPSCs generate neural cell adhesion molecule (NCAM)-positive neurectodermal cells, α-smooth muscle actin (α-SMA)-positive mesodermal cells, and a-fetoprotein (AFP)-positive endodermal cells.

Extracellular Vesicles as a New Feature of Pluripotent Stem Cell Biology

The production of EVs by hiPSCs was confirmed by using electron microscopy.

Extracellular Vesicle Isolation

In order to isolate EVs different approaches were used for: LL-CBMSCs, hESCs and hiPSCs because of the different cell culture conditions.

When LL-CBMSCs reached 80%-90% confluence the culture medium was removed and the cells were washed twice with PBS to remove bovine EVs derived from the serum and aMEM (Sigma-Aldrich) without FBS (Life Technologies) was added (starving step). After 24 hours, EV-containing medium was collected and serially centrifuged to remove dead cells (350 xg for 10 minutes) and cellular debris (5,000 xg for 15 minutes).

EVs were collected with a 3 hour 100,000 xg ultracentrifugation at 4° C. (F37L 8X100 rotor, Sorvall WX 80+ ultracentrifuge; Thermo Fisher Scientific).

Human ESC and human iPSC growth medium did not contain serum, thus the starving step was not needed. hESCs and hiPSCs were cultured in Petri dishes (100 mm) as disclosed above until 80% confluence. Then, 24 hour conditioned medium was collected and serially centrifuged, as described for LL-CBMSCs above.

The supernatant was discarded and EV pellets suspended in 150 μL of PBS, for both LL-CBMSCs and pluripotent stem cells. EVs were stored at −20° C. for subsequent studies.

Nanoparticle Tracking Analysis

To estimate the number and size of isolated EVs, Nanoparticle Tracking Analysis (NTA) was performed using the NanoSight LM10 instrument (NanoSight, Malvern, UK), which exploits light scattering of a laser beam caused by nanoparticle Brownian movements to count EVs.

Isolated EVs were diluted 1:100 in 500 μL of PBS and analyzed. The number of EVs was calculated from the mean of n=5 independent counts on the same sample.

Flow Cytometry Protocol for Extracellular Vesicles

The integrity of EVs was investigated by carboxyfluorescein diacetate succinimidyl ester (CFDA-SE) staining (BD). CFDA-SE is a cell-permeable precursor, which can be converted into a fluorescent molecule, carboxyfluorescein succinimidyl ester (CFSE), by cytoplasmic esterases. In addition, the EV immunophenotype was also assessed. To stain EVs, 50 μL of starved LL-CBMSC- or pluripotent stem cell-conditioned medium were collected. EVs were incubated with 5 μL of 100 μM CFDA-SE for 30 minutes in the dark at RT. Alternatively, EVs were incubated with 10 μL of CD63 (BD), CD81 (BD) or isotype (BD) antibodies for 15 minutes at RT. Right after incubation, 300 μL of PBS were added to each sample and EVs were analyzed immediately. In order to visualize EVs on a FACSCantoll standard cytometer (BD), its physical parameters were pushed to their limit of detection. Moreover, forward scatter (FSC) and side scatter (SSC) were set on a logarithmic scale and the FSC threshold was set to a value of 200-350. The data were analyzed with the FACS Diva version 7 analysis software (BD).

Electron Microscopy

For Scanning Electron Microscopy (SEM), LL-CBMSCs, hESCs and hiPSCs were cultured on glass coverslips, fixed in 2% glutaraldehyde (Sigma-Aldrich) diluted in PBS, washed twice with PBS and stored at 4° C. For Transmission Electron Microscopy (TEM), EVs were collected, resuspended in 200 μL PBS and analyzed within 24 hours. SEM and TEM analyses were performed in collaboration with Università degli Studi dell'Aquila, Dipartimento di Medicina Clinica, Sanità Pubblica, Scienze della Vita e dell'Ambiente.

For SEM analysis, the samples were dehydrated through a graded series of ethanol solutions. Samples were critical-point dried and sputter coated with a SCD040 Balzer Sputterer (Balzers Union, Liechtenstein). A Philips 505 SEM microscope (Philips) was used to examine the samples, using an accelerating voltage of 20 kV. For TEM analysis, the EVs resuspended in PBS were adsorbed to 300 mesh carbon-coated copper grids (Electron Microscopy Sciences) for 5 minutes in a humidified chamber at RT. EVs on grids were then fixed in 2% glutaraldehyde (Sigma-Aldrich) in PBS for 10 minutes and then briefly rinsed with milliQ water. Grids with adhered EVs were examined with a Philips CM 100 TEM microscope (Philips) at 80 kV, after negative staining with 2% phosphotungstic acid (Sigma-Aldrich), brought to pH 7.0 with NaOH. Images were captured by a Kodak digital camera.

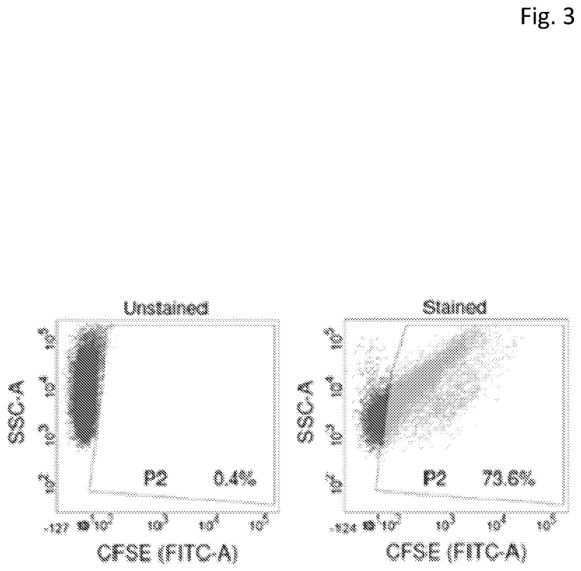

The results show that EVs from hiPSCs were detected as outward structures protruding from the cell surface. Concerning EV secretion, it was located at peripheral protrusions of the plasma membrane, indicating that hiPSCs, as well as LL-CBMSCs, need space to outgrow and release EVs. Afterwards, EV size was assessed by nanoparticle tracking analysis (NTA), which detected a mean size of 256 nm (n=5; standard deviation (SD)=66). Again, this particle size range indicates absence of apoptotic bodies (1-5 μm). Quantification of EVs by NTA revealed that hiPSCs produced a mean of 28 billion EVs per Petri dish (100mm) per day (n=5; SD=12). Integrity of EVs was investigated by carboxyfluorescein diacetate succinimidyl ester (CFDA-SE) staining. Flow cytometry analysis revealed that >70% of the detected events were carboxyfluorescein succinimidyl ester (CFSE) positive, which indicated that they were intact EVs (FIG. 3). In addition, 50% of CFSE-positive EVs also expressed CD63 and CD81 surface markers.

Stem Cell Extracellular Vesicle miRNome Load

A complete miRNome analysis was performed on EVs harvested from hiPSCs, LL-CBMSCs and a pluripotent stem cell control.

The profiling was performed exploiting a Real Time PCR-array system covering 754 human miRNAs, whose sequences were derived from miRBase version 14 database.

miRNA Extraction

Isolated EVs were lysed with 700 μL of QIAzol Lysis Reagent (Qiagen), vortexed and stored at −20° C. until use. To extract miRNA, the miRNeasy Mini Kit (Qiagen) and the RNeasy MinElute Cleanup Kit (Qiagen) were used. Briefly, 140 μL of chloroform were added to the samples, which were shaken vigorously for 15 seconds and let stand for 3 minutes at room temperature (RT). Then, the samples were centrifuged for 15 minutes at 12,000 xg at 8° C. The resulting upper aqueous phase was transferred to a new collection tube, and 350 μL of 70% ethanol were added. After vortexing, the samples were transferred in RNeasy spin columns, placed in 2 mL collection tubes at RT, and centrifuged for 15 seconds at speed speed at 20° C. The filters were discarded and 450 μL of 100% ethanol were added to the miRNA-containing flow-through. Next, 700 μL of samples were transferred to RNeasy MinElute spin columns and centrifuged for 15 seconds at speed speed at 20° C.; the flow-through was discarded. This step was repeated for the remaining 700 μL of samples. Then, 500 μL of RPE Buffer were added to the RNeasy MinElute spin columns and centrifuged for 15 seconds at speed. The flow-through was discarded and the columns were placed into new collection tubes. Next, 500 μL of 80% ethanol were added to the columns and centrifuged for 2 minutes at speed. The flow-through was discarded and the columns were transferred into new collection tubes. An additional centrifugation at speed for 5 minutes was performed with open lids. To elute miRNAs, the columns were transferred into 1.5 mL collection tubes and 20 μL of RNase free-water were added to the filters. The filters were let stand at RT for 5 minutes and then the samples were centrifuged for 1 minute at full speed. RNA quality and quantification were assessed on a 2100 Bioanalyzer instrument (Agilent). The samples were stored at −80° C. until further use.

miRNA Real Time PCR-array

The miRNome analysis was performed in collaboration with Dipartimento di Scienze Cliniche e di Comunità, University of Milan, Milan, Italy. The complete miRNome was investigated using the Real Time PCR TaqMan OpenArray Human MicroRNA Panel array (Thermo Fisher Scientific), covering a total of 754 human miRNAs. The array allowed the simultaneous automated manipulation and analysis of three samples. The experiment was carried out on a QuantStudio 12K Flex Real Time PCR System (Thermo Fisher Scientific). Statistical analysis was performed with R free software version 3.3.1 (https://cran.r-project.org), using VennDiagram package venn.diagram( ) function and gplots package heatmap.2( ) function.

Statistical Analysis

Online interrogation of miRTarBase experimentally-validated miRNA version 7 database was performed using DIANA miRPath version 3 tool (http://snf-515788.vm.okeanos.grnet.gr/), considering KEGG pathway database. The results were merged by pathways union; 0.05 p-value threshold; 0.8 microT threshold; FDR correction on.

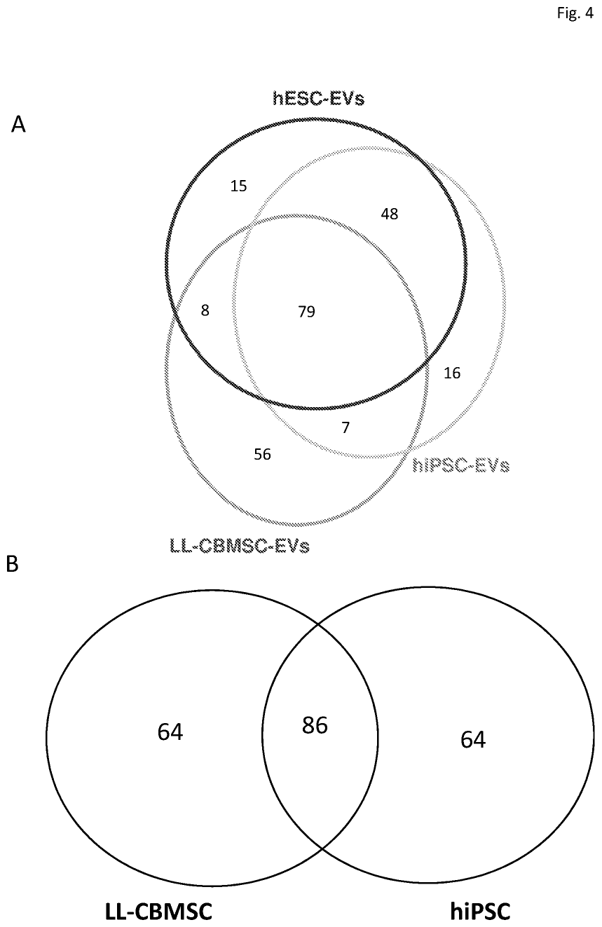

The analysis of stem cell source-unique EV miRNAs was performed on raw PCR amplification data, selecting the 150 sequences (top 150) with the highest output signal. Interestingly, the majority of miRNAs present in LL-CBMSC-EVs were also found in hiPSC-EVs (86 common miRNAs—FIG. 4A), consistently with the pluripotent control. Only 16 miRNAs were found to be exclusively incorporated into hiPSC-EVs, while LL-CBMSC-EVs showed 56 specific miRNA sequences (FIG. 4A). Focusing on the ten most incorporated miRNAs, hiPSC-EVs showed a pattern identical to that of the pluripotent control EVs (Table 6).

TABLE 6
Top 10 incorporated miRNAs
EV source

| LL-CBMSCs | hESCs | hiPSCs |
|---|---|---|
| miR-24 | miR-367 | miR-367 |
| miR-125b | miR-302b | miR-20a |
| miR-221 | miR-20a | miR-302a |
| miR-222 | miR-302a | miR-302d |
| miR-100 | miR-19b | miR-92a |
| miR-99a | miR-92a | miR-302b |
| miR-31 | miR-106a | miR-19b |
| miR-21 | miR-17 | miR-106a |
| miR-1274B | miR-302c | miR-302c |
| miR-20a | miR-302d | miR-17 |

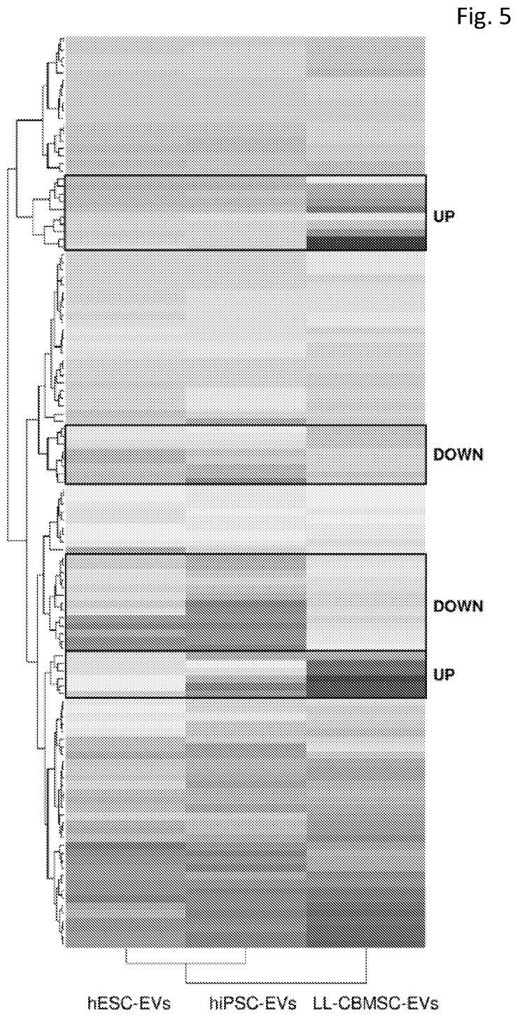

Fold change data of the miRNAs plotted in a heatmap, by R statistical software, revealed two classes of more incorporated (UP) and two classes of less incorporated (DOWN) hiPSC-EV miRNAs (FIG. 5). The cellular pathways targeted by UP and DOWN classes of differentially expressed miRNAs were addressed by interrogation of the miRTarBase database, which uses only experimentally-validated miRNAs and are summarized in Table 7.

TABLE 7
Top 5 significant pathways related to the
differentially-expressed miRNAs

| KEGG pathways | | KEGG pathways | |
|---|---|---|---|
| UP | p-value | DOWN | p-value |
| Protactin | 1.2^{∧}( [symbol] 6) | Fatty acid | <1^{∧}( [symbol] 325) |
| signaling pathway | | biosynthesis | |
| Writ signaling | 2.5^{∧}( [symbol] 5) | Steroid | <1^{∧}( [symbol] 325) |
| pathway | | biosynthesis | |
| Endocytosis | 0.0010 | ECM-receptor | <1^{∧}( [symbol] 325) |
| | | interaction | |
| Oocyte meiosis | 0.0138 | Fatty acid metabolism | 1.1^{∧}( [symbol] 16) |
| Signaling | 0.0183 | Mucin type | 1.4^{∧}( [symbol] 7) |
| pathways regulating | | O-Glycan | |
| pluripotency of | | biosynthesis | |
| stem cells | | | |

[symbol]

indicates data missing or illegible when filed

The results show that the UP classes are involved, among others, in regulation of stem cell pluripotency and in Wnt signaling pathway (key for pluripotent stem cell specification and proliferation). The DOWN classes revealed major associations with fatty acid metabolism and with adherence features. These data are consistent with the stemness properties of hiPSCs and LL-CBMSCs, since 1) pluripotent stem cell metabolism relies more on glycolysis than fatty acid oxidation, and 2) MSCs express a wide range of adhesion molecules.

To conclude, even though the heatmap-associated hierarchical clustering analysis grouped hiPSC-EVs and the pluripotent control together, this was due to differences in the EV incorporation relative amount. Notably, the EV miRNome load was largely maintained following reprogramming, since the majority of the top 150 miRNAs were common between the hiPSC-EVs and LL-CBMSC-EVs.

The reprogramming of long living (LL)-cord blood (CB) multipotent mesenchymal stromal cells (MSCs) was proved to be feasible. Interestingly, no feeder cells were used, guaranteeing a more straightforward and unbiased xeno-free process. This was due to the feeder properties of non-reprogrammed LL-CBMSCs in culture. Moreover, a minimal immunophenotype panel composed of HLA-ABC and CD73 was proposed in order to assess successful MSC reprogramming. The induced pluripotent stem cell (hiPSC) lines generated were characterized successfully for stemness and pluripotency features, indicating that a complete reprogramming process was achieved.

Importantly for this research and for a possible therapeutic application of hi PSC-EVs, reprogramming preserved secretion of extracellular vesicles with physical properties similar to those of parental LL-CBMSCs. Noteworthy, the EV miRNome load was largely maintained, which represents a prerequisite to envision a switch from LL-CBMSC-EV to hi PSC-EV use.

### EXAMPLE II

Extracellular Vesicle-Driven Damage Recovery

In order to study EV potential in acute brain ischemia treatment, an ex vivo model was exploited. This kind of model has the advantage of higher control over the experimental conditions and less variability. In addition, it is more suitable to study the mechanism of action of a therapeutic approach, compared to in vivo models of sensorimotor and cognitive deficits, because molecular and cellular events may be investigated at the tissue and cellular level (e.g.: signaling pathways, cell viability, tissue secretion in response to damage).

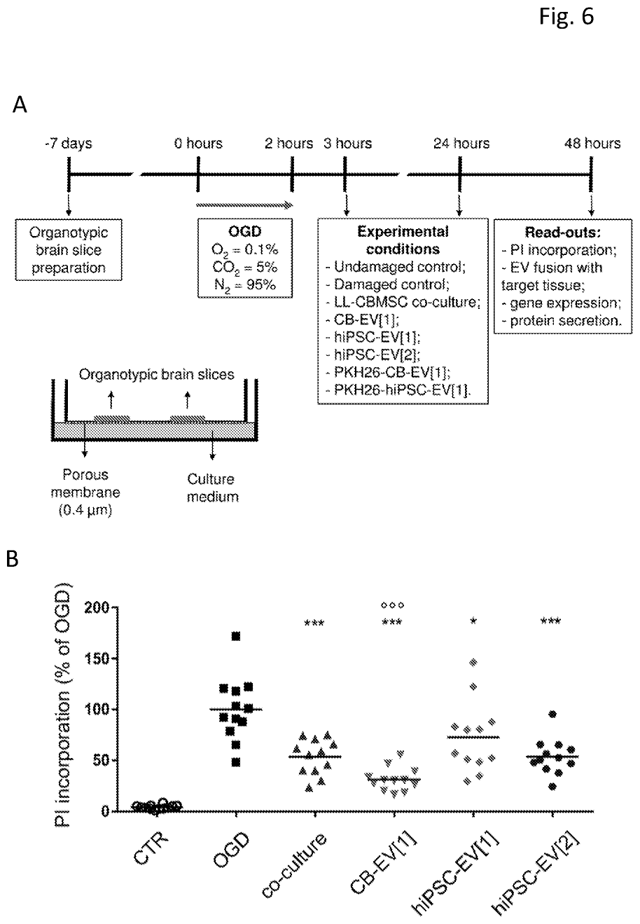

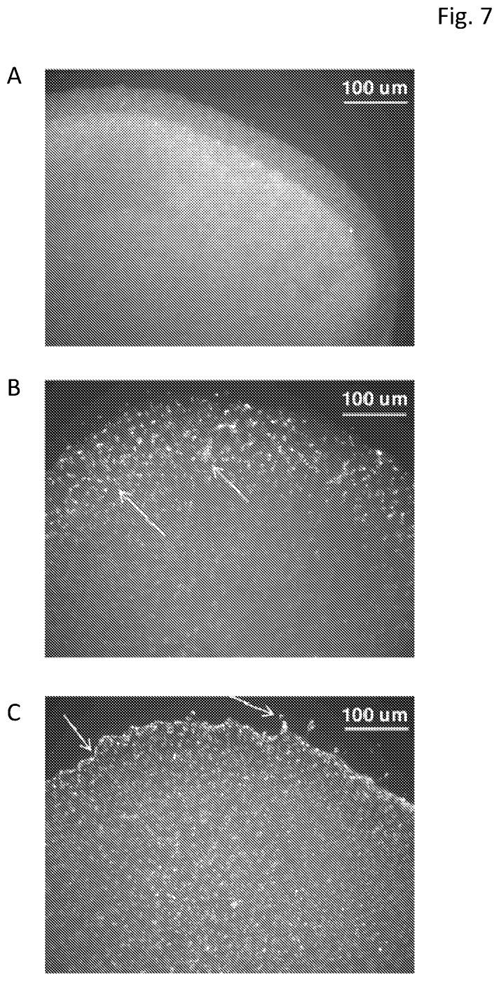

Ischemic damage was induced in organotypic mouse brain slices by a standard oxygen and glucose deprivation (OGD) culture condition. The complete experimental schematic is summarized in FIG. 6. This insult mimics the sudden lack of oxygen and nutrients in the ischemic brain, which leads to metabolic and oxidative stress, and eventually to inflammation and tissue necrosis. Hence, all these different aspects of ischemia-driven injury were addressed to investigate if EV-based treatments could be of any therapeutic use.

Animals

Experimental procedures involving animals and their care were conducted in conformity with the institutional guidelines at the IRCCS Institute for

Pharmacological Research “Mario Negri” in compliance with national (Decreto Legge nr 116/92, Gazzetta Ufficiale, supplement 40, Feb. 18, 1992; Circolare nr 8, Gazzetta Ufficiale, Jul. 14, 1994) and international laws and policies (EEC Council Directive 86/609, OJL 358, 1, Dec. 12, 1987; Guide for the Care and Use of Laboratory Animals, U.S. National Research Council (Eighth Edition) 2011). Mice (Harlan Laboratories) were housed in a specific pathogen-free vivarium (room temperature (RT) 21±1° C., 12 hour light-dark cycle, free access to food and water). All efforts were made to minimize animal suffering and to reduce the number of animals used.

Mouse Brain Slice Preparation

Organotypic cortical brain slices (named cortical slices from now on) were obtained from prefrontal cortex of C57BL/6 mouse pups (P1-3). Mouse pup brains were removed from the skull under sterile conditions and were immersed into a 3% agar solution. Tissue blocks containing mesencephalic and forebrain levels were dissected out, fixed onto a specimen stage of a vibratome (VT 1000S; Leica Biosystems) with Super Attack glue, and placed in ice-cold (4° C.) artificial cerebral spinal fluid (ACSF) solution (NaCl=87 mM, NaHCO₃=25 mM, NaH₂PO₄=1.25 mM, MgCl₂=7 mM, CaCl₂=0.5 mM, KCl=2.5 mM, D-glucose=25 mM, sucrose=75 mM, Penicillin=50 U/ml, Streptomicin=50 μg/ml, equilibrated with 95% O₂ and 5% CO₂, pH=7.4). Prefrontal cortex coronal sections of 200 μm thickness were cut. Cortical slices were transferred into Petri dishes filled with ice-cold ACSF. Only intact cortical slices were placed on membranes of tissue culture inserts (0.4 μm pore size Millicell Culture insert; Merk Millipore) with two slices per insert and placed in 6-well plate wells, each filled with 1 mL of culture medium (MEM-Glutamax 25%, basal medium eagle 25% (Life Technologies), horse serum 25% (Euroclone), glucose 0.6%, penicillin 100 U/mL, streptomicin 100 μg/mL (Euroclone); pH=7.2). All the cultures were maintained at 37° C. in 5% CO₂. After two days, the incubation medium was changed with neurobasal medium (Life Technologies) supplemented with B27 (1:50) (Life Technologies), L-glutamine (1:100) (Life Technologies), penicillin (100 U/mL), streptomycin (100 μg/mL) (Euroclone) (NB/B27 medium) and replaced there after every two days.

Ischemic Insult

After one week in culture, cortical slices were subjected to oxygen and glucose deprivation (OGD), an in vitro model of brain ischemia. The culture medium was removed, cortical slices were washed twice with PBS (Euroclone) and transferred into a temperature-controlled (37±1° C.) hypoxic chamber (InvivO₂400; Baker Ruskinn) at [O₂]=0.1%, [CO₂]=5%, [N₂]=95%. Once in the hypoxic chamber, the PBS was replaced with deoxygenated glucose-free medium. After a 2 hour OGD insult, cortical slices were transferred to a normoxic incubator and the medium was replaced with NB/B27 medium. Control cortical slices, not exposed to ischemic injury, were maintained in normoxic incubator with NB/B27 medium. The rescue experiment was conducted according to the experimental plan summarized in FIG. 6.

Propidium Iodide Incorporation

To evaluate cell death 48 hours after injury, the inserts with cortical slices were moved to new plates and fresh NB/B27 medium containing 2 μM of propidium iodide (PI; Sigma-Aldrich) was added. After a 30 minute incubation, images were captured at ×4 magnification, using the TRITC filter of an Olympus IX71 microscope (Olympus). Images were then analyzed with Fiji software (University of Wisconsin-Madison) for quantification of PI-positive cells.

Extracellular Vesicle Labeling

EVs suspended in Diluent C (Sigma-Aldrich) were mixed with PKH26 (Sigma-Aldrich) and incubated for 20 minutes at RT in the dark. The reaction was stopped by adding an equal volume of 1% BSA (Sigma-Aldrich). EVs were then ultracentrifuged at 100,000 xg for 1 hour and suspended in PBS (Euroclone). PBS that received the same treatment as above was used as negative control. Cortical slices, which received labeled EVs and PBS, were observed after 24 hours. Images were captured at ×4 magnification, using the TRITC filter of an Olympus IX71 microscope (Olympus).

Briefly, the experimental conditions are the following:
Undamaged brain slices (positive control, n=12);
OGD-damaged brain slices (negative control, n=12);
LL-CBMSCs in co-culture with damaged brain slices (co-culture, n=12);
LL-CBMSC-EV treated damaged brain slices, one-fold dose (CB-EV[1], n=12);
hiPSC-EV treated damaged brain slices, one-fold dose (hiPSC-EV[1], n=12);
hiPSC-EV treated damaged brain slices, two-fold dose (hiPSC-EV[2], n=12);
PKH26-labelled LL-CBMSC-EV treated damaged brain slices (n=2);
PKH26-labelled hiPSC-EV treated damaged brain slices (n=2).

The one-fold dose consisted in 2.4 billion EVs per brain slice and it was determined based on an estimation of EV production by LL-CBMSCs in co-culture. A direct comparison between LL-CBMSC- and hiPSC-EVs was planned in order to assess if and how much reprogramming had affected EV protective properties. To detect any dose-response phenomenon, the two-fold dose was also used. EV administration was repeated for two subsequent days, based on the assumption that LL-CBMSCs in co-culture would constantly generate new EVs. In addition, EV membranes were labelled with PKH26 to investigate if fusion with the target tissue had taken place during the treatments.

At 48 hours post-OGD insult, the effectiveness of the applied treatments was addressed by the evaluation of cell necrosis that is the endpoint of the ischemic cascade. Necrotic cells in the brain tissue were quantified by propidium iodide (PI) incorporation. As summarized in FIG. 6B, all the treatments rescued cell mortality, showing statistically significant reductions in PI signal, measured by fluorescence microscopy. In particular, all stem cell EV-based treatments demonstrated a neuroprotective effect equal to that of LL-CBMSC co-culture. The most effective treatment among them was CB-EV[1], which resulted in a statistically significant lower PI incorporation only if compared to hiPSC-EV[1]. Nonetheless, hi PSC-EVs possessed relevant neuroprotective properties, which were more evident for hiPSC-EV[2].

At 48 hours post-OGD, PKH26-labelled EV presence within the brain slices was addressed by fluorescence microscopy (FIG. 7). The EV signals were detected as bright fluorescent spots, located especially at tissue slice borders.

Real Time qRT-PCR Analysis

The next step was to evaluate which cell type was damaged by the OGD insult and if the treatments could have a beneficial effect on them. The presence of specific neural cell types was assessed at the transcriptional level by Real Time qRT-PCR analysis performed on markers typical of neurons (Map2), endothelial cells (Cd31), microglia (Cd11b), and astrocytes (Gfap).

Gene Expression

Forty-eight hours after injury, cortical slices were collected and total RNA was extracted by miRNeasy mini kit (Qiagen). The samples were treated with DNase (Life Technologies) and reverse-transcribed with random hexamer primers using Multi-Scribe Reverse Transcriptase (Life Technologies). Real time reverse transcription quantitative PCR was performed and relative gene expression determined by the ΔΔCt method; β-Act, Rpl27 and B2m were used as house-keeping genes. Data are expressed as log₂ of the fold difference compared to the control group. Genes and primer sequences are reported in Table 8; the primer pairs were designed not to amplify human sequences.

TABLE 8
Primer sequences

| Gene | Forward Primer | SEQ ID NO | Reverse Primer | SEQ ID NO |
|---|---|---|---|---|
| β-act | CGCGAGCACAG | SEQ ID NO: 226 | GCAGCGATATCG | SEQ ID NO: |
| | CTTCTTT | | TCATCCAT | 239 |
| Rpl27 | TCATGAAACCC | SEQ ID NO: 227 | GAGGTGCCATCG | SEQ ID NO: |
| | GGGAAAGT | | TCAATGT | 240 |
| B2m | CTGACCGGCCT | SEQ ID NO: 228 | TATGTTCGGCTTC | SEQ ID NO: |
| | GTATGCTAT | | CCATTCT | 241 |
| Map2 | TCAGCTGACAG | SEQ ID NO: 229 | TTGTGTTGGGCT | SEQ ID NO: |
| | AGAAACAGCA | | TCCTTCTC | 242 |
| Cd31 | GTCGTCCATGT | SEQ ID NO: 230 | GCACAGGACTCT | SEQ ID NO: |
| | CCCGAGAA | | CGCAATCC | 243 |
| Gfap | GAAACCGCATC | SEQ ID NO: 231 | TCGGATGGAGGT | SEQ ID NO: |
| | ACCATTCC | | TGGAGA | 244 |
| Cd1lb | GAGCAGCACTG | SEQ ID NO: 232 | ATACGACTCCTG | SEQ ID NO: |
| | AGATCCTGTTTA | | CCCTGGAA | 245 |
| | A | | | |
| Bdnf | AGGCACTGGAA | SEQ ID NO: 233 | AAGGGCCCGAAC | SEQ ID NO: |
| | CTCGCAATG | | ATACGATT | 246 |
| Vegf | CATTCCTGGCC | SEQ ID NO: 234 | TGGTTGGAACCG | SEQ ID NO: |
| | CTGAGTCAA | | GCATCTTT | 247 |
| | TCGC | | | |
| Bcl2 | GTGCCTGTGGT | SEQ ID NO: 235 | CCTGTGCCACTT | SEQ ID NO: |
| | CATGGATCTG | | GCTCTTTAG | 248 |
| Bax | GAGAGGCAGCG | SEQ ID NO: 236 | TGCTCGATCCTG | SEQ ID NO: |
| | GCAGTGAT | | GATGAAACC | 249 |
| Mki67 | GATAACGCCAC | SEQ ID NO: 237 | ATGGATGCTCTC | SEQ ID NO: |
| | CGAGGACAA | | TTCGCAGG | 250 |
| Pcna | ACCTTTGAAGAT | SEQ ID NO: 238 | GCAGCGATATCG | SEQ ID NO: |
| | TGCTCCTGAGA | | TCATCCAT | 251 |

Evaluation of Metabolic and Oxidative Stress

To investigate levels of metabolites and parameters of oxidative stress in cortical slice conditioned medium, colorimetric or fluorescence reaction-based assays were used for ATP (Abcam), adenosine (Biovision), glutamate (Fitzgerald Industries International), malondialdehyde (Fitzgerald Industries International), superoxide dismutase (Biovision) and glutathione (Fitzgerald Industries International).

Statistical Analysis

All statistical analyses were performed using Prism software (GraphPad). Online interrogation of miRTarBase experimentally-validated mi RNA version 7 database was performed using DIANA miRPath version 3 tool (http://snf-515788.vm.okeanos.grnet.gr/), considering KEGG pathway database. The results were merged by pathways union; 0.05 p-value threshold; 0.8 microT threshold; FDR correction on.

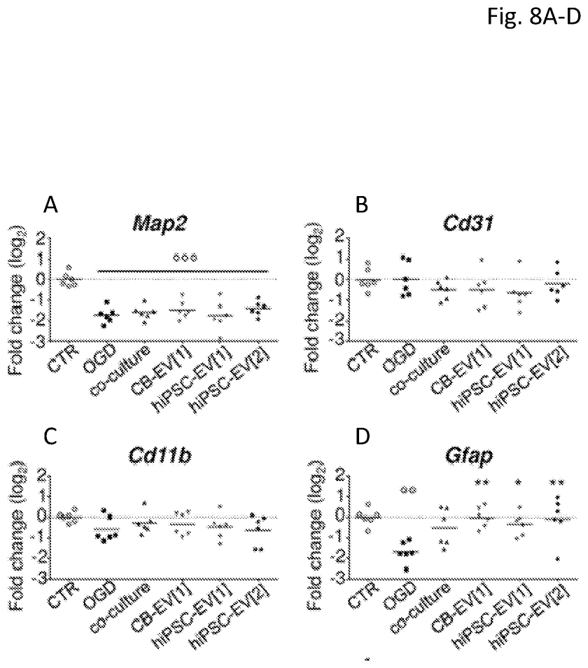

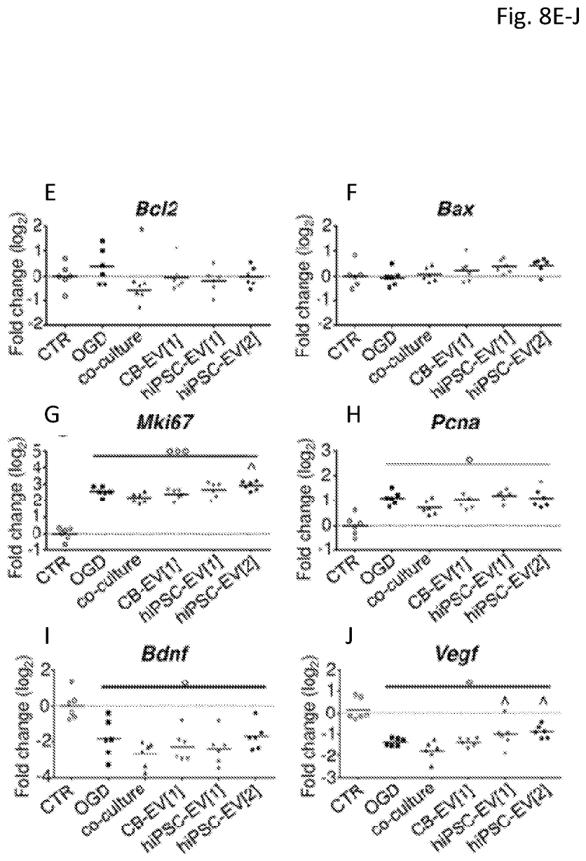

Neurons and astrocytes were affected by the OGD insult, as shown by a statistically significant reduction of the respective marker mRNA transcripts, whereas endothelial cells and microglia did not (FIG. 8A-D). Importantly, astrocyte marker mRNA level was rescued in all treatments, indicating that this cell type is the main therapeutic target of stem cells and stem cell-EVs. Notably, the co-culture treatment showed only a partial and not significant rescue.

The expression of anti-apoptotic (Bcl2) and pro-apoptotic (Bax) genes was also investigated to evaluate if a modulation of apoptosis by the treatments had played a role in the observed neuroprotection. Unfortunately, no treatment revealed increased expression of Bcl2 or decreased expression of Bax, compared to untreated control (FIGS. 8E-F). The same approach was used to address if the treatments promoted proliferation, but, also in this case, no significant fluctuations in Mki67 and Pcna gene expression was observed, compared to OGD negative control (FIGS. 8G-H). Instead, undamaged positive control brain slices show statistically significant lower Mki67 and Pcna mRNA levels, compared to the other experimental conditions. This suggested that in this ex vivo model the OGD insult per se, followed by reoxygenation and glucose availability, induced transcription of these genes, maybe as an endogenous response to injury. Yet, a significant difference was identified between the co-culture condition, compared to hiPSC-EV[2], hinting at a higher stimulation of proliferation by the latter treatment. The gene expression of growth factors crucial for ischemia-triggered injury recovery was also assessed: brain-derived neurotrophic factor (Bdnf) and vascular endothelial growth factor A (Vegfa). The transcript levels were significantly lower than those of the undamaged positive control. Notwithstanding, both hiPSC-EV[1] and [2] treatments demonstrated significantly higher Vegfa mRNA levels, compared to the co-culture condition (FIGS. 8I-J).

Tissue Modulation as Stem Cell Extracellular Vesicle Mechanism of Action

Based on the previously assessed anti-inflammatory properties of LL-CBMSCs, which could be relevant in the context of brain ischemia, modulation of inflammatory cytokines secreted by brain slices in the culture medium was investigated by multiplexed ELISA. The levels of INFγ, IL1β, IL2, IL6, IL10, IL12p70, IL17 and TNFα were simultaneously measured for positive and negative controls, and for co-culture, CB-EV[1] and hiPSC-EV[2] treatments.

Multiplex Quantification of Inflammatory Factors

The Cytokine 1 mouse Cyraplex assay (Aushon BioSystems, Billerica, Mass., USA) was used to detect inflammatory cytokines present in cortical slice conditioned medium. The assay allowed the simultaneous quantification of murine TNFα, INFγ, IL1β, IL2, IL6, IL10, IL12p70 and IL17. The cell culture supernatants were centrifuged at 350 xg for 10 minutes to remove dead cells, and then they were stored at −80° C. The samples were thawed completely and mixed by gently vortexing, before performing the assay. In addition, unconditioned NB/B27 medium was used as blank. All the samples were used undiluted and diluted 1:10 in Sample Diluent (Aushon BioSystems) Lyophilized analyte standards were reconstituted in Sample Diluent (Aushon BioSystems) and standard curves were prepared by serial dilution, following manufacturer's instructions. Briefly, the MicroClime Lid (Aushon BioSystems) was filled with distilled water and stored until use. The assay plate was washed four times with 300 μL of wash buffer (Aushon BioSystems), then it was dried by firmly patting on absorbent paper. Next, 50 μL of standards or sample were pipetted into each well. The plate was covered with the MicroClime Lid (Aushon BioSystems) and then it was incubated for 3 hours at RT on a plate shaker set at 60 rpm. Another wash step was performed as previously described. Next, 50 μL of Biotinylated Antibody Reagent (Aushon BioSystems) were added to each well. The plate was covered with the MicroClime Lid (Aushon BioSystems) and then it was incubated for 30 minutes at RT on a plate shaker set at 60 rpm. A final washing step was performed as previously described. The wells were then filled with Block/Stabilizing Solution (Aushon BioSystems) and incubated for 30 minutes at RT. Next, the solution in excess was removed, avoiding to dry completely the wells. The final steps for imaging and analysis were performed in double-blind. The detection of chemiluminescence was performed on the Cyrascan instrument (Aushon BioSystems) and analyzed by the Cyrasoft software (Aushon BioSystems).

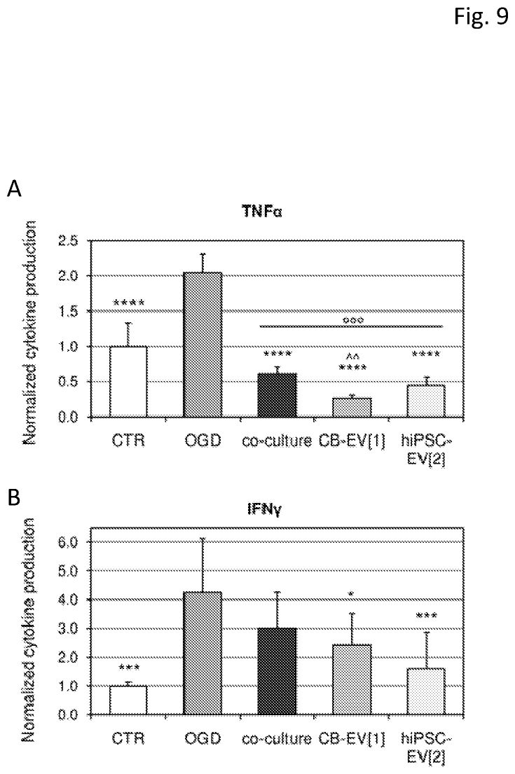

The results are summarized in FIG. 9 and show that detectable levels of secreted protein were found for TNFα and IFNγ. This was a very relevant result, because microglia-produced TNFα is one major mediator of post-ischemic early phase inflammation and it is responsible for many aspects of it. Furthermore, CD4⁺ Th1 lymphocyte-produced IFNγ is involved in late phase ischemic events, and it is a potent inducer of TNFα, thus promoting amplification and maintenance of neuroinflammation. Going into details, TNFα and INFγ secreted protein levels underwent statistically significant increases in OGD condition, compared to undamaged control (FIGS. 9A-B). All treatment conditions showed a statistically significant reduction of TNFα, compared to OGD condition. The effect was so dramatic as to significantly decrease the protein levels below the undamaged control condition by a two-fold change. In addition, CB-EV[1] treatment showed a significant lower reduction of TNFα, compared to the co-culture condition. Concerning INFγ, decreased levels of secreted protein were observed in all treatments, compared to the negative control; statistically significant results were obtained for CB-EV[1] and for hiPSC-EV[2], with the latter demonstrating a stronger action, but not for the co-culture condition. Both TNFα and INFγ results indicated that complete rescue of these two crucial ischemia-driven injury parameters was achieved by the EV-based treatments.

In conclusion, these data show that EVs from LL-CBMSC and hiPSC are able to effectively exert neuroprotection in an organotypic ex vivo model of acute brain ischemia, reducing considerably the entity of tissue necrosis. The mechanism unveiled in this context counteracts brain injury via the modulation of the damaged tissue environment to establish a pro-regenerative milieu. Noteworthy, the ex vivo model mimicked very well an acute phase brain ischemia inflammatory response. This confirmed its validity to assess the efficacy of the developed EV-based treatments in the very early stages of the pathology. In particular, the action of EVs happens trough TNFα, a pleiotropic peptide involved in inflammation- and immune-related activities, which plays a role not only in ischemia, but also in brain trauma and cerebral infection. In addition, EV-based treatments targeted also Interferon y, whose capacity to activate innate and adaptive immune responses can act in concert with TNFα to establish chronic inflammation through a positive regulatory loop found in many neurological diseases.

1-41. (canceled)

42. Extracellular vesicles (EVs) derived from Mesenchymal Stem Cells of Cord Blood (CB-MSCs) or from induced Pluripotent Stem Cells derived from CB-MSCs (CB-MSC-iPSCs), wherein said EVs are characterized by the expression of at least 5 miRNA selected from: SEQ ID NO: 2, 4, 6, 8, 16, 17, 18, 25, 26, 28, 29, 31, 32, 34, 37, 38, 44, 50, 62, 77, 80, 87, 94, 103, 108, 110, 114, 136, 164, 169, 172, 174.

43. The EVs according to claim 42, wherein said CB-MSCs are a subtype of CB-MSCs named Long Living-Cord Blood Mesenchymal Stem Cells (LL-CB-MSCs) and the CB-MSC-iPSCs are a cell line of human induced Pluripotent Stem Cells (hiPSC) CBMSC304-hiPSC deposited on Oct. 26^{th} 2017 at the Leibniz-lnstitut DSMZ with Accession Number DSM ACC3332.

44. The EVs according to claim 43, wherein the EVs derived from LL-CB-MSCs are characterized by the expression of at least 5 or all miRNAs, selected from: SEQ ID NO: 4, 6, 28, 29, 31, 32, 34, 50, 87, 94.

45. The EVs according to claim 43, wherein the EVs derived from hiPSC DSM ACC3332 are characterized by the expression of at least 5 or all miRNAs, selected from: SEQ ID NO: 2, 18, 26, 28, 44, 62, 77, 169, 172, 174.

46. The EVs according to claim 42, wherein the EVs are characterized by the expression of SEQ ID NO: 4, 8, 25, 164, said miRNA targeting TNFα mRNA; and by the expression of : SEQ ID NO: 16, 17, 34, 37, 38, 80, 103, 108, 110, 114, 136, said miRNA targeting INFγ mRNA.

47. The EVs according to claim 42, wherein the EVs are characterized by the expression of at least one of the following combination of miRNAs: the combination SEQ ID N: 1-79 and/or the combination SEQ ID NO: 80-86 and/or the combination SEQ ID NO: 87-94 and/or the combination SEQ ID NO: 95-150 and/or the combination SEQ ID NO: 151-198 and/or the combination SEQ ID NO: 199-214.

48. The EVs according to claim 42, wherein the EVs are characterized by the expression of SEQ ID NO: 1-214.

49. The EVs according to claim 42 characterized by the expression of CD63 (CD63⁺) and/or CD81 (CD81⁺).

50. The EVs according to claim 42, wherein said LL-CB-MSCs are MSCs isolated from human cord blood, and are characterized by: 1) high maximum cumulative population doublings (CPD) not less than 15; and/or 2) at least one the following marker: CD90⁺, CD105⁺, CD73⁺, NG2⁺, CD146⁺, PDGFRβ⁺, CD56⁺, CD45⁻, CD34⁻, CD271⁻; and/or 3) proliferate even under low density-seeding conditions around 3-10 cells/cm².

51. A signature of markers wherein said markers comprise at least five miRNA or all miRNAs, selected from: SEQ ID NO: 2, 4, 6, 8, 16, 17, 18, 25, 26, 28, 29, 31, 32, 34, 37, 38, 44, 50, 62, 77, 80, 87, 94, 103, 108, 110, 114, 136, 164, 169, 172, 174, or least 5 or all miRNAs, selected from: SEQ ID NO: 4, 6, 28, 29, 31, 32, 34, 50, 87, 94; or at least five or all miRNAs, selected from: SEQ ID NO: 2, 18, 26, 28, 44, 62, 77, 169, 172, 174.

52. A method of using the EVs according to claim 42 to treat or prevent ischemia or inflammation, or to regenerate and/or stimulate and/or repair a body area after ischemia, or to treat or prevent a side effect or a symptom or a drawback caused by or associated with ischemia or inflammation, comprising a step of administering the EVs to a patient in need thereof.

53. The method according to claim 52, wherein said side effect or symptom or drawback caused by or associated with ischemia is secondary brain injury and/or reperfusion injury, and wherein said side effect or symptom or drawback caused by or associated with ischemia is selected from: tissue pain, tissue heat, tissue redness, tissue swelling, tissue loss of function, release of inflammatory mediators, vasodilation, increased vessel permeability, recruitment of immune system cells, migration of immune system cells, activation of the complement system, systemic inflammation and any combination thereof.

54. The method according to claim 52, wherein said body area is selected from: connective tissue, vasculature, epithelium, endothelium, the nervous tissue, astrocytes, oligodendrocytes, microglia, ependymal cells, brain, pancreas, intestine, skeletal muscles, heart, lung, limbs, kidney, liver, eyes, skin, spleen, thymus, bones, tendons, musculoskeletal grafts, corneae, heart valves, nerves, veins, the central and/or peripheral nervous system.

55. A method for inducing Pluripotent Stem Cells said method comprising the following steps:
isolating MSCs of human cord blood (CB-MSCs);
reprogramming said CB-MSCs by introducing and expressing into said cells, by using a non-integrating viral-based system, the following reprogramming factors OCT4, SOX2, KLF4, cMYC or L-MYC, wherein said CB-MSCs are a subtype CB-MSCs named LL-CB-MSCs characterized by: 1) high maximum CDP not less than 15; and/or 2) at least one, the following marker: CD90⁺, CD105⁺, CD73⁺, NG2⁺, CD146⁺, PDGFRβ⁺, CD56⁺, CD45⁻, CD34⁻, CD271⁻ wherein (+) means positive and (−) means negative; and/or 3) proliferate even under low density-seeding conditions around 3-10 cells/cm².

56. A cell line of human induced Pluripotent Stem Cells CBMSC304-hiPSC deposited on Oct. 26th, 2017 at the Leibniz-lnstitut DSMZ-Deutsche Sammlung von Mikroorganlsmen and Zellkulturen GmbH with Accession Number DSM ACC3332.

57. The EVs according to claim 42, wherein said LL-CB-MSCs are MSCs isolated from human cord blood, and are characterized by: 1) a CPD not less than 30; and/or 2) the following marker(s): CD90⁺, CD105⁺, CD73⁺, NG2⁺, CD146⁺, PDGFRβ⁺, CD56⁺, CD45⁻, CD34⁻, CD271.

58. The method according to claim 52, wherein the inflammation is mediated by TNF-α and/or IGF-γ.

59. The method according to claim 55, wherein said LL-CB-MSCs are MSCs isolated from human cord blood, and are characterized by: 1) a CPD not less than 30; and/or 2) the following marker(s): CD90⁺, CD105⁺, CD73⁺, NG2⁺, CD146⁺, PDGFRβ⁺, CD56⁺, CD45⁻, CD34⁻, CD271⁻².

60. The method according to claim 52, wherein said body area is brain.

61. A culture media comprising the EVs of claim 42 and media for proliferation of cells.

62. A pharmaceutical composition comprising the EVs of claim 42 and a pharmaceutically acceptable excipient.
